(19) United States
(12) Patent Application Publication
(10) Pub. No.: US 2008/0146645 A1
(43) Pub. Date: Jun. 19, 2008

(54) Process for Preparing Indolinone Phenylaminopropanol Derivatives

(75) Inventors: Anita Wai-Yin Chan, Fort Lee, NJ (US); Zhixian Ding, Fort Lee, NJ (US); Mousumi Ghosh, Elmwood Park, NJ (US); Mahmut Levent, Bloomfield, NJ (US); Panolil Raveendranath, Monroe, NY (US); Jianxin Ren, Nanuet, NY (US); Maotang Zhou, Cedar Knolls, NJ (US); Asaf Alimardanov, Nanuet, NY (US); Alexander V. Gontcharov, Rivervale, NJ (US); Antonia A. Nikitenko, Tarrytown, NY (US); John R. Potoski, West Nyack, NY (US); Girija Raveendranath, Monroe, NY (US); Vijay Raveendranath, Albany, NY (US); Sanjay Raveendranath, Monroe, NY (US)

Correspondence Address: WOODCOCK WASHBURN LLP/WYETH, CIRA CENTRE, 12TH FLOOR, 2929 ARCH STREET, PHILADELPHIA, PA, 19104-2891, US

(73) Assignee: Wyeth, Madison, NJ (US)

(21) Appl. No.: 11/843,364

(22) Filed: Aug. 22, 2007

Related U.S. Application Data
(60) Provisional application No. 60/839,978, filed on Aug. 24, 2006.

(51) Int. Cl. C07D 209/34; A61K 31/4015

(52) U.S. Cl. 514/418; 548/486

(57) ABSTRACT

Processes are disclosed for preparing indolinone phenylaminopropanol derivatives, particularly chiral indolinone phenylaminopropanol derivatives of the general formula:

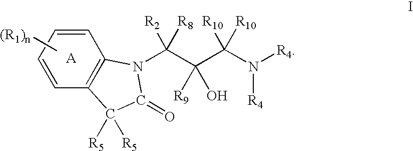

The processes disclosed may be used to prepare, inter alia, 7-fluoro-1-[(1S, 2R)-1-(3-fluorophenyl)-2-hydroxy-3-(methylamino)propyl]-3,3-dimethyl-1,3-dihydro-2H-indol-2-one and 7-fluoro-1-[(1S, 2R)-1-(3,5-difluorophenyl)-2-hydroxy-3-(methylamino)propyl]-3,3-dimethyl-1,3-dihydro-2H-indol-2-one. Intermediates of the processes are also disclosed.

### CROSS REFERENCE TO RELATED APPLICATIONS

This application claims the benefit of U.S. Application No. 60/839,978 filed Aug. 24, 2006, the entire disclosure of which is incorporated herein by reference.

### FIELD OF THE INVENTION

The present invention relates to processes for preparing indolinone phenylaminopropanol derivatives, particularly chiral indolinone phenylaminopropanol derivatives.

### BACKGROUND OF THE INVENTION

Certain indolinone phenylaminopropanol derivatives, such as those disclosed in US-A1-2005/0222148 (the disclosure of which is hereby incorporated herein by reference in its entirety), including 7-fluoro-1-[(1S,2R)-1-(3-fluorophenyl)-2-hydroxy-3-(methyl amino)propyl]-3,3-dimethyl-1,3-dihydro-2H-indol-2-one (Example 101), are useful in preventing and treating conditions ameliorated by monoamine reuptake including, inter alia, vasomotor symptoms (VMS), sexual dysfunction, gastrointestinal and genitourinary disorders, chronic fatigue syndrome, fibromyalgia syndrome, nervous system disorders, and combinations thereof, particularly those conditions selected from the group consisting of major depressive disorder, vasomotor symptoms, stress and urge urinary incontinence, fibromyalgia, pain, diabetic neuropathy, and combinations thereof.

US 2005/0222142 and US 2005/0222148 disclose that the compounds of formula I of this invention, including 7-fluoro-1-[(1S,2R)-1-(3-fluorophenyl)-2-hydroxy-3-(methyl amino)propyl]-3,3-dimethyl-1,3-dihydro-2H-indol-2-one, may be generally prepared from compounds of formula 4 via Scheme II. This route generally involves the selective protection of the primary alcohol followed by conversion of the secondary alcohol to a leaving group. The publication discloses that conventional methods for the selective protection of a primary alcohol, and conventional methods for converting of a secondary alcohol into a leaving group could be used for this conversion. In accordance with the preferred embodiment, compounds of formula 4 were treated with para-nitrobenzoyl chloride in pyridine at low temperature (preferably below about 0° C.) to form compounds of formula 11. Compounds of formula 11 were converted to a secondary mesylate of formula 12 via reaction with methanesulfonyl chloride in dichloromethane using triethylamine as base. The reaction was preferably carried out at temperatures between about −15° C. and about 10° C. Deprotection of the primary alcohol in compounds of formula 12 allowed for the formation of a primary epoxide through an S_{N}2 reaction resulting in an inversion of the stereocenter. It was disclosed that any conventional method for deprotection of a primary alcohol, and any conventional method for epoxide formation onto an alpha leaving group could be employed for this conversion. In accordance with the preferred embodiment, compounds of formula 12 were treated with an aqueous solution of a suitable base in organic solvent, preferably, aqueous sodium hydroxide in dioxane. The resulting epoxide of formula 13 was ring-opened regioselectively with an amine to produce the desired aminoalcohol of formula I-b. It was also disclosed that any conventional method for the regioselective ring opening of a primary epoxide could be employed for this conversion. In accordance with the preferred embodiment, compounds of formula 13 were treated with an excess of an alcoholic amine solution in a sealed flask, either at room temperature or heated to about 40° C. to about 90° C. It was further disclosed that compounds of formula I-b were converted into a pharmaceutically acceptable salt using conventional methods.

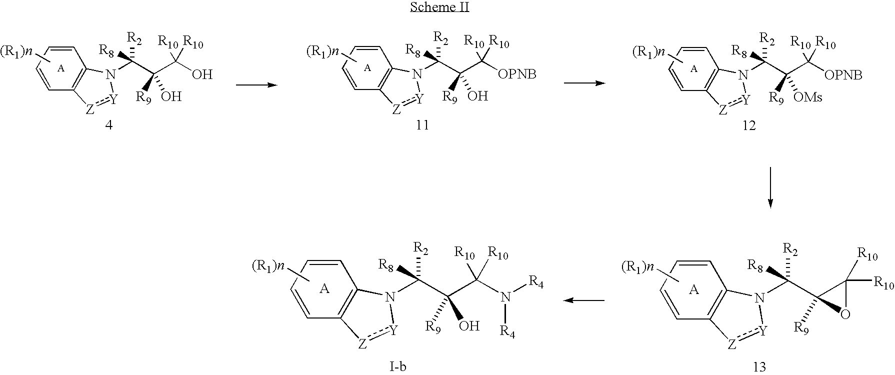

### Where:

A, Y, Z, R₁, n, R₂, and R₄, R₈, R₁₀ are as previously described R₉ is H
PNB=para-nitrobenzoyl or any conventional protecting group; and
OMs=methanesulfonate or any conventional leaving group

US 2005/0222142 and US 2005/0222148 disclose that compounds of formula 4 may generally be formed via a regio- and stereo-selective ring opening of an appropriately substituted epoxide of formula 17 (formed via an epoxidation of an appropriately substituted allylic alcohol) with an appropriately substituted compound of formula 16 (Scheme IV). It was disclosed that any conventional method for the regio- and stereo-selective ring opening of an epoxide could be employed for this conversion. In accordance with the preferred embodiment of this invention, compounds of formula 16 were treated with a base, e.g. sodium hydride, sodium tert-butoxide, potassium hydroxide, potassium tert-butoxide or potassium hydroxide, then treated with the epoxide of formula 17. The epoxide of formula 17 could be pre-treated with a Lewis acid, e.g. titanium iso-propoxide, boron-trifluoride, etc. to ensure regio-selective ring-opening. The reaction occurred at room temperature over a duration of about 2 hours to about 72 hours. Alternatively, compounds of formula 16 that are suitably nucleophilic, e.g. indoline, could be heated with the epoxide of formula 17 at temperatures from about 50° C. to about 170° C. to form compounds of formula 4.

US 2005/0222142 and US 2005/0222148 disclose that epoxidation of trans-allylic alcohols may be performed either racemically or asymmetrically using methods described in the literature. In accordance with the preferred embodiment, racemic epoxidation was conducted with either peracetic acid or meta-chloroperbenzoic acid. The publications describe that if it were desired to produce a single enantiomer of compounds of formula I, asymmetric epoxidation of an allylic alcohol could be performed with tert-butylhydroperoxide or cumene hydroperoxide in the presence of the appropriate tartrate ester, titanium (IV) isopropoxide, and molecular sieves, as is well established in the literature (e.g. K. B. Sharpless, et. al., J. Org. Chem. 1986, 51, 3710). Compounds of formula 16 and the starting allylic alcohols were either available from commercial sources or were accessible through methods well established in the literature.

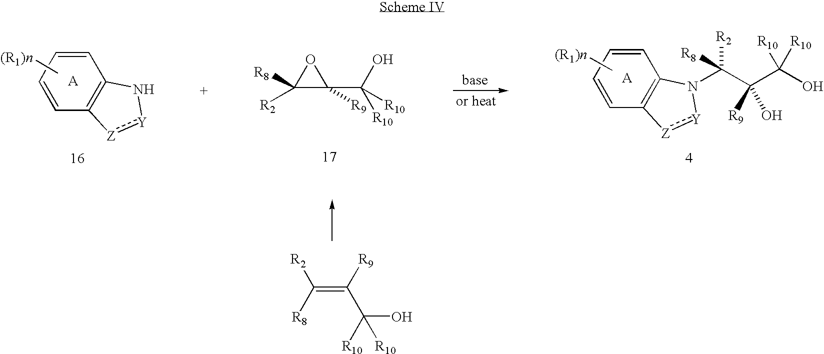

### Where:

A, Y, Z, R₁, n, R₈, R₉, R₁₀ and R₂ are as previously described.

While these methods are suitable for preparing the compounds of formula I, (such as 7-fluoro-1-[(1S,2R)-1-(3-fluorophenyl)-2-hydroxy-3-(methyl amino)propyl]-3,3-dimethyl-1,3-dihydro-2H-indol-2-one (Example 101), for example) on a laboratory scale, they are not suitable for larger scale syntheses. For example, one problem is that the methods require an abundance of chromatographic purifications of the intermediates because most of them are oils. Another problem is that there is a very inefficient opening with the sodium salt of the indolinone. Yet another problem includes a low total for the conversion of the diol (4) to the amino alcohol (I-b).

Thus, there is a ongoing need for more facile and higher yielding processes for preparing indolinone phenylaminopropanol derivatives, particularly chiral indolinone phenylaminopropanol derivatives, including 7-fluoro-1-[(1S,2R)-1-(3-fluorophenyl)-2-hydroxy-3-(methyl amino)propyl]-3,3-dimethyl-1,3-dihydro-2H-indol-2-one, useful for, inter alia, preventing and treating conditions ameliorated by monoamine reuptake including, e.g., vasomotor symptoms (VMS), sexual dysfunction, gastrointestinal and genitourinary disorders, chronic fatigue syndrome, fibromyalgia syndrome, nervous system disorders, and combinations thereof. The present invention is directed to processes for preparing such indolinone phenylaminopropanol derivatives, particularly chiral indolinone phenylaminopropanol derivatives, including 7-fluoro-1-[(1S,2R)-1-(3-fluorophenyl)-2-hydroxy-3-(methyl amino)propyl]-3,3-dimethyl-1,3-dihydro-2H-indol-2-one, for these and other important uses.

### SUMMARY OF THE INVENTION

The present invention is generally directed to processes for preparing indolinone phenylaminopropanol derivatives, including 7-fluoro-1-[(1S,2R)-1-(3-fluorophenyl)-2-hydroxy-3-(methylamino)propyl]-3,3-dimethyl-1,3-dihydro-2H-indol-2-one. This invention is also directed to various intermediates useful in the preparation of these indolinone phenylaminopropanol derivatives, and the methods of preparing such intermediates. Method described can be used for the preparation of the other enantiomer and diastereomers.

In some embodiments, the present invention is directed to processes for preparing indolinone phenylaminopropanol compounds, comprising the steps of:

a. coupling a compound of formula IV:

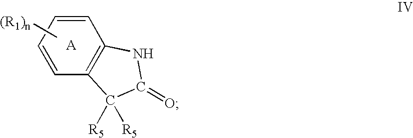

or a metal salt thereof;

with a compound of formula II:

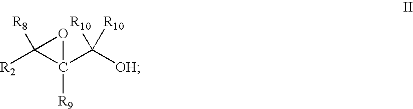

to form a diol compound of formula V:

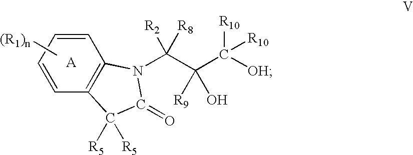

wherein said coupling is carried out in the presence of:
an optional Lewis acid catalyst;
a solvent composition comprising at least one polar, aprotic solvent; and
an excess of a strong non-nucleophilic base selected from the group consisting of RₓRₓ—N-M, R_{y}—O-M, and R_{y}—Mg—X;
where:
each Rₓ is independently alkyl substituted with 0-3 R₁, aryl substituted with 0-3 R₁, or (R_{z})₃Si;
or said Rₓ groups, together with the N atom to which they are attached, form a cyclic amine;
R_{y} is alkyl substituted with 0-3 R₁;
R_{z} is R₁;
M is Na, Li, or K;
X is Cl, Br, or I;
provided that said strong non-nucleophilic base is other than sodium t-butoxide;
wherein:
R₁ is, independently at each occurrence, alkyl, alkoxy, halo, CF₃, OCF₃, arylalkyloxy substituted with 0-3 R₁₁, aryloxy substituted with 0-3 R₁₁, aryl substituted with 0-3 R₁₁, heteroaryl substituted with 0-3 R₁₁, hydroxy, alkanoyloxy, nitro, nitrile, alkenyl, alkynyl, alkylsulfoxide, phenylsulfoxide substituted with 0-3 R₁₁, alkylsulfone, phenylsulfone substituted with 0-3 R₁₁, alkylsulfonamide, phenylsulfonamide substituted with 0-3 R₁₁, heteroaryloxy substituted with 0-3 R₁₁, heteroarylmethyloxy substituted with 0-3 R₁₁, alkylamido, or arylamido substituted with 0-3 R₁₁; or two adjacent R₁ also represent methylenedioxy;
R₂ is aryl substituted with 0-3 R₁ or heteroaryl substituted with 0-3 R₁;
R₅ is, independently at each occurrence, H, C₁-C₄ alkyl, aryl substituted with 0-3 R₁, or cyano; or the two R₅ form a carbocyclic ring of 3-7 carbons;
R₈ is H, or C₁-C₄ alkyl;
R₉ is H, or C₁-C₄ alkyl;
R₁₀ is, independently at each occurrence, H, or C₁-C₄ alkyl;
R₁₁ is alkyl, alkoxy, halo, CF₃, OCF₃, hydroxy, alkanoyloxy, nitro, nitrile, alkenyl, alkynyl, alkylsulfoxide, alkylsulfone, alkylsulfonamide, or alkylamido; or two adjacent R₁₁ also represent methylenedioxy;
n is an integer from 0 to 4; and
wherein 1-3 carbon atoms in ring A may optionally be replaced with N.

In other embodiments, the processes further comprising the step of:
b. selectively activating the terminal hydroxy group of said diol compound of formula V with a compound of the formula (R₁₂SO₂)₂O or R₁₂SO₂Z with or without the use of catalyst in the presence of an optional base in an inert solvent to form a compound of formula Va:

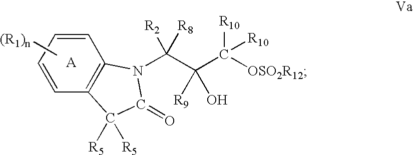

wherein:
Z is Cl or Br; and
R₁₂ is alkyl substituted with 0-3 R₁ or aryl substituted with 0-3 R₁.

In yet other embodiments, the processes further comprising the step of:
c. converting said compound of formula Va in the presence of a base and an optional phase transfer catalyst to a compound of formula VI:

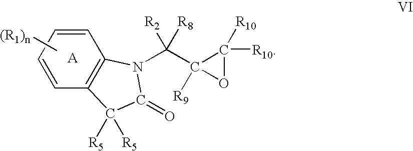

In alternative embodiments, the processes further comprising the step of:
c. treating said compound of formula V with phosphine and dialkyl azodicarboxylate in inert solvent to form a compound of formula VI:

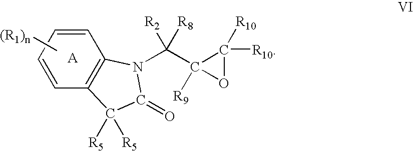

In other alternative embodiments, the processes further comprising the step of:
c. treating said compound of formula V with phosphine and dialkyl azodicarboxylate in inert solvent to form a compound of formula VI:

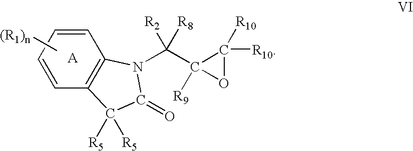

In other embodiments, the processes further comprising the step of:
d. reacting said compound of formula VI with NHR₄R₄ with optional Lewis acid catalyst in an optional polar solvent to form a compound of formula I:

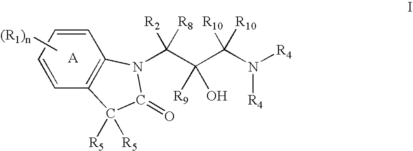

wherein:
R₄ is, independently at each occurrence, H, C₁-C₄ alkyl, arylalkyl, heteroarylmethyl, cycloheptylmethyl, cyclohexylmethyl, cyclopentylmethyl, or cyclobutylmethyl; and
with respect to the compound of formula I, R₁₀ and R₄, together with the nitrogen to which R₄ is attached, form a nitrogen-containing ring containing 3 to 6 carbons.

In yet further embodiments, the processes further comprising the step of:

e. forming a pharmaceutically acceptable salt of said compound of formula 1.

In other embodiments, the invention is directed to processes, comprising the step of:

aa. transesterifying a diol compound of formula V:

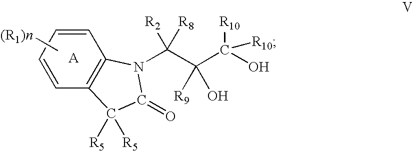

including a diol compound of formula V*

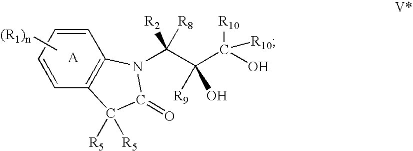

with a trialkyl orthoacetate in the presence of a catalytic amount of an acid or an acid catalyst to form a cyclic orthoester compound of formula XI:

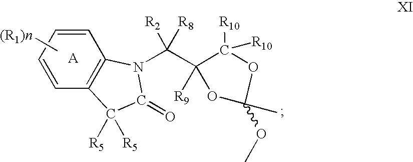

including a cyclic orthoester compound of formula XI*:

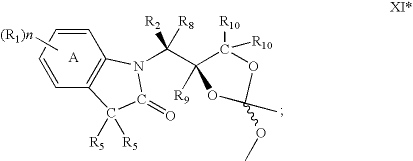

wherein:
R₁ is, independently at each occurrence, alkyl, alkoxy, halo, CF₃, OCF₃, arylalkyloxy substituted with 0-3 R₁₁, aryloxy substituted with 0-3 R₁₁, aryl substituted with 0-3 R₁₁, heteroaryl substituted with 0-3 R₁₁, hydroxy, alkanoyloxy, nitro, nitrile, alkenyl, alkynyl, alkylsulfoxide, phenylsulfoxide substituted with 0-3 R₁₁, alkylsulfone, phenylsulfone substituted with 0-3 R₁₁, alkylsulfonamide, phenylsulfonamide substituted with 0-3 R₁₁, heteroaryloxy substituted with 0-3 R₁₁, heteroarylmethyloxy substituted with 0-3 R₁₁, alkylamido, or arylamido substituted with 0-3 R₁₁; or two adjacent R₁ also represent methylenedioxy;
R₂ is aryl substituted with 0-3 R₁ or heteroaryl substituted with 0-3 R₁;
R₅ is, independently at each occurrence, H, C₁-C₄ alkyl, aryl substituted with 0-3 R₁, or cyano; or the two R₅ form a carbocyclic ring of 3-7 carbons;
R₈ is H, or C₁-C₄ alkyl;
R₉ is H, or C₁-C₄ alkyl;
R₁₀ is, independently at each occurrence, H, or C₁-C₄ alkyl;
R₁₁ is alkyl, alkoxy, halo, CF₃, OCF₃, hydroxy, alkanoyloxy, nitro, nitrile, alkenyl, alkynyl, alkylsulfoxide, alkylsulfone, alkylsulfonamide, or alkylamido; or two adjacent R₁₁ also represent methylenedioxy;
n is an integer from 0 to 4; and
wherein 1-3 carbon atoms in ring A may optionally be replaced with N.

In some embodiments, the present invention is directed to isolated, solid forms of a compound of formula V:

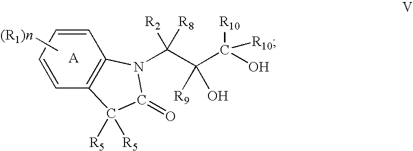

wherein:

R₁ is, independently at each occurrence, alkyl, alkoxy, halo, CF₃, OCF₃, arylalkyloxy substituted with 0-3 R₁₁, aryloxy substituted with 0-3 R₁₁, aryl substituted with 0-3 R₁₁, heteroaryl substituted with 0-3 R₁₁, hydroxy, alkanoyloxy, nitro, nitrile, alkenyl, alkynyl, alkylsulfoxide, phenylsulfoxide substituted with 0-3 R₁₁, alkylsulfone, phenylsulfone substituted with 0-3 R₁₁, alkylsulfonamide, phenylsulfonamide substituted with 0-3 R₁₁, heteroaryloxy substituted with 0-3 R₁₁, heteroarylmethyloxy substituted with 0-3 R₁₁, alkylamido, or arylamido substituted with 0-3 R₁₁; or two adjacent R₁ also represent methylenedioxy;

R₂ is aryl substituted with 0-3 R₁ or heteroaryl substituted with 0-3 R₁;

R₅ is, independently at each occurrence, H, C₁-C₄ alkyl, aryl substituted with 0-3 R₁, or cyano; or the two R₅ form a carbocyclic ring of 3-7 carbons;

R₈ is H, or C₁-C₄ alkyl;

R₉ is H, or C₁-C₄ alkyl;

R₁₀ is, independently at each occurrence, H, or C₁-C₄ alkyl;

R₁₁ is alkyl, alkoxy, halo, CF₃, OCF₃, hydroxy, alkanoyloxy, nitro, nitrile, alkenyl, alkynyl, alkylsulfoxide, alkylsulfone, alkylsulfonamide, or alkylamido; or two adjacent R₁₁ also represent methylenedioxy;

n is an integer from 0 to 4; and

wherein 1-3 carbon atoms in ring A may optionally be replaced with N.

In yet other embodiments, the present invention is directed to compounds of formula VI:

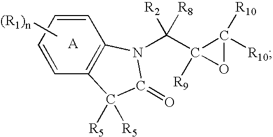

including an epoxide compound of formula VI*:

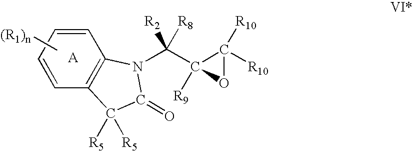

wherein:

R₁ is, independently at each occurrence, alkyl, alkoxy, halo, CF₃, OCF₃, arylalkyloxy substituted with 0-3 R₁₁, aryloxy substituted with 0-3 R₁₁, aryl substituted with 0-3 R₁₁, heteroaryl substituted with 0-3 R₁₁, hydroxy, alkanoyloxy, nitro, nitrile, alkenyl, alkynyl, alkylsulfoxide, phenylsulfoxide substituted with 0-3 R₁₁, alkylsulfone, phenylsulfone substituted with 0-3 R₁₁, alkylsulfonamide, phenylsulfonamide substituted with 0-3 R₁₁, heteroaryloxy substituted with 0-3 R₁₁, heteroarylmethyloxy substituted with 0-3 R₁₁, alkylamido, or arylamido substituted with 0-3 R₁₁; or two adjacent R₁ also represent methylenedioxy;

R₂ is aryl substituted with 0-3 R₁ or heteroaryl substituted with 0-3 R₁;

R₅ is, independently at each occurrence, H, C₁-C₄ alkyl, aryl substituted with 0-3 R₁, or cyano; or the two R₅ form a carbocyclic ring of 3-7 carbons;

R₈ is H, or C₁-C₄ alkyl;

R₉ is H, or C₁-C₄ alkyl;

R₁₀ is, independently at each occurrence, H, or C₁-C₄ alkyl;

R₁₁ is alkyl, alkoxy, halo, CF₃, OCF₃, hydroxy, alkanoyloxy, nitro, nitrile, alkenyl, alkynyl, alkylsulfoxide, alkylsulfone, alkylsulfonamide, or alkylamido; or two adjacent R₁₁ also represent methylenedioxy;

n is an integer from 0 to 4; and

wherein 1-3 carbon atoms in ring A may optionally be replaced with N.

In other embodiments, the invention is directed to product produced by the processes described above.

In another embodiment, the invention is directed to compositions, comprising:

a compound of formula I; and

less than about 35% by weight, based on the total weight of the composition, of a compound of formula I′:

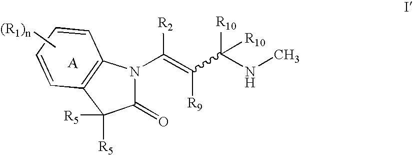

R₁ is, independently at each occurrence, alkyl, alkoxy, halo, CF₃, OCF₃, arylalkyloxy substituted with 0-3 R₁₁, aryloxy substituted with 0-3 R₁₁, aryl substituted with 0-3 R₁₁, heteroaryl substituted with 0-3 R₁₁, hydroxy, alkanoyloxy, nitro, nitrile, alkenyl, alkynyl, alkylsulfoxide, phenylsulfoxide substituted with 0-3 R₁₁, alkylsulfone, phenylsulfone substituted with 0-3 R₁₁, alkylsulfonamide, phenylsulfonamide substituted with 0-3 R₁₁, heteroaryloxy substituted with 0-3 R₁₁, heteroarylmethyloxy substituted with 0-3 R₁₁, alkylamido, or arylamido substituted with 0-3 R₁₁; or two adjacent R₁ also represent methylenedioxy;

R₂ is aryl substituted with 0-3 R₁ or heteroaryl substituted with 0-3 R₁;

R₅ is, independently at each occurrence, H, C₁-C₄ alkyl, aryl substituted with 0-3 R₁, or cyano; or the two R₅ form a carbocyclic ring of 3-7 carbons;

R₉ is H, or C₁-C₄ alkyl;

R₁₀ is, independently at each occurrence, H, or C₁-C₄ alkyl;

R₁₁ is alkyl, alkoxy, halo, CF₃, OCF₃, hydroxy, alkanoyloxy, nitro, nitrile, alkenyl, alkynyl, alkylsulfoxide, alkylsulfone, alkylsulfonamide, or alkylamido; or two adjacent R₁₁ also represent methylenedioxy;

n is an integer from 0 to 4;

wavy line represents both stereochemical configurations between the carbons to which R₉ and R₁₀ are attached; and

wherein 1-3 carbon atoms in ring A may optionally be replaced with N.

### DETAILED DESCRIPTION OF ILLUSTRATIVE EMBODIMENTS

The present invention is generally directed to processes for preparing indolinone phenylaminopropanol derivatives, including 7-fluoro-1-[(1S,2R)-1-(3-fluorophenyl)-2-hydroxy-3-(methylamino)propyl]-3,3-dimethyl-1,3-dihydro-2H-indol-2-one and pharmaceutically acceptable salts thereof. This invention is also directed to various intermediates useful in the preparation of these indolinone phenylaminopropanol derivatives, and the processes of preparing such intermediates. The processes described can be used for the preparation of the other enantiomer and diastereomers. The indolinone phenylaminopropanol derivatives are useful, alone, or in compositions, for the prevention and treatment of conditions ameliorated by monoamine reuptake including, inter alia, vasomotor symptoms (VMS), sexual dysfunction, gastrointestinal and genitourinary disorders, chronic fatigue syndrome, fibromyalgia syndrome, nervous system disorders, and combinations thereof, particularly those conditions selected from the group consisting of major depressive disorder, vasomotor symptoms, stress and urge urinary incontinence, fibromyalgia, pain, diabetic neuropathy, and combinations thereof.

The following definitions are provided for the full understanding of terms and abbreviations used in this specification.

As used herein and in the appended claims, the singular forms “a,” “an,” and “the” include the plural reference unless the context clearly indicates otherwise. Thus, for example, a reference to “an antagonist” includes a plurality of such antagonists, and a reference to “a compound” is a reference to one or more compounds and equivalents thereof known to those skilled in the art, and so forth.

The abbreviations in the specification correspond to units of measure, techniques, properties, or compounds as follows: “min” means minutes, “h” means hour(s), “μL” means microliter(s), “mL” means milliliter(s), “mM” means millimolar, “M” means molar, “mmole” means millimole(s), “cm” means centimeters, “SEM” means standard error of the mean and “IU” means International Units. “Δ° C.” and A “ED₅₀ value” means dose which results in 50% alleviation of the observed condition or effect (50% mean maximum endpoint).

“Norepinephrine reuptake inhibitor” is abbreviated NRI.

“Serotonin reuptake inhibitor” is abbreviated SRI.

“Norepinephrine” is abbreviated NE.

“Serotonin is abbreviated 5-HT.

The terms “component,” “composition of compounds,” “compound,” “drug,” or “pharmacologically active agent” or “active agent” or “medicament” are used interchangeably herein to refer to a compound or compounds or composition of matter which, when administered to a subject (human or animal) induces a desired pharmacological and/or physiologic effect by local and/or systemic action.

The terms “component”, “drug” or “pharmacologically active agent” or “active agent” or “medicament” are used interchangeably herein to refer to a compound or compounds or composition of matter which, when administered to an organism (human or animal) induces a desired pharmacologic and/or physiologic effect by local and/or systemic action.

The term “modulation” refers to the capacity to either enhance or inhibit a functional property of a biological activity or process, for example, receptor binding or signaling activity. Such enhancement or inhibition may be contingent on the occurrence of a specific event, such as activation of a signal transduction pathway and/or may be manifest only in particular cell types. The modulator is intended to comprise any compound, e.g., antibody, small molecule, peptide, oligopeptide, polypeptide, or protein, preferably small molecule, or peptide.

As used herein, the term “inhibitor” refers to any agent that inhibits, suppresses, represses, or decreases a specific activity, such as serotonin reuptake activity or the norepinephrine reuptake activity.

The term “inhibitor,” as used herein, is intended to comprise any compound, e.g., antibody, small molecule, peptide, oligopeptide, polypeptide, or protein, preferably small molecule or peptide, that exhibits a partial, complete, competitive and/or inhibitory effect on mammalian, preferably the human norepinephrine reuptake or both serotonin reuptake and the norepinephrine reuptake, thus diminishing or blocking, preferably diminishing, some or all of the biological effects of endogenous norepinephrine reuptake or of both serotonin reuptake and the norepinephrine reuptake.

Within the present invention, the compounds of formula I may be prepared in the form of pharmaceutically acceptable salts. As used herein, the term “pharmaceutically acceptable salts” refers to salts prepared from pharmaceutically acceptable non-toxic acids, including inorganic salts, and organic salts. Suitable non-organic salts include inorganic and organic acids such as acetic, benzenesulfonic, benzoic, camphorsulfonic, citric, ethanesulfonic, fumaric, gluconic, glutamic, hydrobromic, hydrochloric, isethionic, lactic, malic, maleic, mandelic, methanesulfonic, mucic, nitric, pamoic, pantothenic, phosphoric, succinic, sulfuric, tartaric acid, p-toluenesulfonic and the like. Particularly preferred are hydrochloric, hydrobromic, phosphoric, and sulfuric acids, and most preferably is the hydrochloride salt.

The term “alkyl,” as used herein, refers to an aliphatic hydrocarbon chain of 1 to about 20 carbon atoms, preferably 1 to 10 carbon atoms, more preferably, 1 to 6 carbon atoms, and even more preferably, 1 to 4 carbon atoms and includes straight and branched chains such as methyl, ethyl, n-propyl, isopropyl, n-butyl, isobutyl, sec-butyl, t-butyl, n-pentyl, isopentyl, neo-pentyl, n-hexyl, and isohexyl. Lower alkyl refers to alkyl having 1 to 4 carbon atoms. Alkyl groups can be optionally substituted. The optional substituent or substituents may be 1 to 3 members selected from the group consisting of C₁-C₆ alkyl, halogen, C₂-C₇ alkenyl, C₂-C₇ alkynyl, C₃-C₈ cycloalkyl, aralkyl, aryl optionally substituted with R₇, heterocycle optionally substituted with R, hydroxy, C₁-C₆ alkoxy, aryl-oxy, oxo (═O), —CN, —C(═O)H, —CO₂H, —OCO₂C₁-C₆ alkyl, —CO₂C₁-C₆ alkyl, —CO₂-aryl, —CO₂(C₁-C₆ alkyl)aryl, —OCO₂-aryl, —C(═O)NH₂, —C(═O)NHOH, amino, alkylamino, dialkylamino, —NHC(═O)NH—C₁-C₆ alkyl, —NHSO₂—C₁-C₆ alkyl, —NHSO₂-aryl, and —NHSO₂-heterocycle, where R is halo, C₁-C₆ alkoxy, C₁-C₆ alkyl, C₂-C₇ alkenyl, C₂-C₇ alkynyl, hydroxy, —C(═O)C₁-C₇ alkyl, —SO₂—C₁-C₆ alkyl, —CO₂—C₁-C₆ alkyl, C₂₋₇ acyl, or alkoxycarbonylalkyl.

The term “alkoxy,” as used herein, refers to the group R—O— where R is an alkyl group of 1 to 6 carbon atoms.

The term “alkoxycarbonyl,” as used herein, refers to the group R—O—C(═O)— where R is an alkyl group of 1 to 6 carbon atoms.

The term “alkanoyl,” as used herein, refers to the group R—C(═O)— where R is an alkyl group of 1 to 6 carbon atoms.

The term “alkanoyloxy,” as used herein, refers to the group R—C(═O)—O— where R is an alkyl group of 1 to 6 carbon atoms.

The term “alkylaminocarbonyl,” as used herein, refers to the group R—NH—C(═O)— where R is an alkyl group of 1 to 6 carbon atoms.

The term “alkylcarbonylamino,” as used herein, refers to the group R—C(═O)—NH where R is an alkyl group of 1 to 6 carbon atoms.

The term “alkenyl” or “olefinic,” as used herein, refers to an alkyl group of at least two carbon atoms having one or more double bonds, wherein alkyl is as defined herein. Alkenyl groups preferably contain 2 to about 20 carbon atoms, more preferably 2 to 10 carbon atoms, even more preferably, 2 to 6 carbon atoms, and yet even more preferably, 2 to 4 carbon atoms. Alkenyl groups can be optionally substituted. The optional substituent or substituents may be 1 to 3 members selected from the group consisting of C₁-C₆ alkyl, halogen, C₂-C₇ alkenyl, C₂-C₇ alkynyl, C₃-C₈ cycloalkyl, aralkyl, aryl optionally substituted with R₇, heterocycle optionally substituted with R, hydroxy, C₁-C₆ alkoxy, aryl-oxy, oxo (═O), —CN, —C(═O)H, —CO₂H, —OCO₂C₁-C₆ alkyl, —CO₂C₁-C₆ alkyl, —CO₂-aryl, —CO₂(C₁-C₆ alkyl)aryl, —OCO₂-aryl, —C(═O)NH₂, —C(═O)NHOH, amino, alkylamino, dialkylamino, —NHC(═O)NH—C₁-C₆ alkyl, —NHSO₂—C₁-C₆ alkyl, —NHSO₂-aryl, and —NHSO₂-heterocycle, where R is halo, C₁-C₆ alkoxy, C₁-C₆ alkyl, C₂-C₇ alkenyl, C₂-C₇ alkynyl, hydroxy, —C(═O)C₁-C₇ alkyl, —SO₂—C₁-C₆ alkyl, —CO₂—C₁-C₆ alkyl, C₂₋₇ acyl, or alkoxycarbonylalkyl.

The term “alkynyl,” as used herein, refers to an alkyl group of at least two carbon atoms having one or more triple bonds, wherein alkyl is as defined herein. Alkynyl groups preferably contain 2 to about 20 carbon atoms, more preferably 2 to 10 carbon atoms, even more preferably, 2 to 6 carbon atoms, and yet even more preferably, 2 to 4 carbon atoms. Alkynyl groups can be optionally substituted. The optional substituent or substituents may be 1 to 3 members selected from the group consisting of C₁-C₆ alkyl, halogen, C₂-C₇ alkenyl, C₂-C₇ alkynyl, C₃-C₈ cycloalkyl, aralkyl, aryl optionally substituted with R₇, heterocycle optionally substituted with R, hydroxy, C₁-C₆ alkoxy, aryl-oxy, oxo (═O), —CN, —C(═O)H, —CO₂H, —OCO₂C₁-C₆ alkyl, —CO₂C₁-C₆ alkyl, —CO₂-aryl, —CO₂(C₁-C₆ alkyl)aryl, —OCO₂-aryl, —C(═O)NH₂, —C(═O)NHOH, amino, alkylamino, dialkylamino, —NHC(═O)NH—C₁-C₆ alkyl, —NHSO₂—C₁-C₆ alkyl, —NHSO₂-aryl, and —NHSO₂-heterocycle, where R is halo, C₁-C₆ alkoxy, C₁-C₆ alkyl, C₂-C₇ alkenyl, C₂-C₇ alkynyl, hydroxy, —C(═O)C₁-C₇ alkyl, —SO₂—C₁-C₆ alkyl, —CO₂—C₁-C₆ alkyl, C₂₋₇ acyl, or alkoxycarbonylalkyl.

The term “aryl” as used herein, refers to an optionally substituted, mono-, di-, tri-, or other multicyclic aromatic ring system having from about 5 to about 50 carbon atoms (and all combinations and subcombinations of ranges and specific numbers of carbon atoms therein), with from about 6 to about 10 carbons being preferred. Non-limiting examples include, for example, phenyl, naphthyl, anthracenyl, and phenanthrenyl. The optional substituent or substituents may be 1 to 3 members selected from the group consisting of C₁-C₆ alkyl, halogen, C₂-C₇ alkenyl, C₂-C₇ alkynyl, C₃-C₈ cycloalkyl, aralkyl, aryl optionally substituted with R₇, heterocycle optionally substituted with R, hydroxy, C₁-C₆ alkoxy, aryl-oxy, oxo (═O), —CN, —C(═O)H, —CO 2H, —OCO₂C₁-C₆ alkyl, —CO₂C₁-C₆ alkyl, —CO₂-aryl, —CO₂(C₁-C₆ alkyl)aryl, —OCO₂-aryl, —C(═O)NH₂, —C(═O)NHOH, amino, alkylamino, dialkylamino, —NHC(═O)NH—C₁-C₆ alkyl, —NHSO₂—C₁-C₆ alkyl, —NHSO₂-aryl, and —NHSO₂-heterocycle, where R is halo, C₁-C₆ alkoxy, C₁-C₆ alkyl, C₂-C₇ alkenyl, C₂-C₇ alkynyl, hydroxy, —C(═O)C₁-C₇ alkyl, —SO₂—C₁-C₆ alkyl, —CO₂—C₁-C₆ alkyl, C₂₋₇ acyl, or alkoxycarbonylalkyl.

The term “heteroaryl,” as used herein, refers to an optionally substituted, mono-, di-, tri-, or other multicyclic aromatic ring system that includes at least one, and preferably from 1 to about 4 sulfur, oxygen, or nitrogen heteroatom ring members. Heteroaryl groups can have, for example, from about 3 to about 50 carbon atoms (and all combinations and subcombinations of ranges and specific numbers of carbon atoms therein), with from about 4 to about 10 carbons being preferred. Non-limiting examples of heteroaryl groups include, for example, pyrryl, furyl, pyridyl, 1,2,4-thiadiazolyl, pyrimidyl, thienyl, isothiazolyl, imidazolyl, tetrazolyl, pyrazinyl, pyrimidyl, quinolyl, isoquinolyl, thiophenyl, benzothienyl, isobenzofuryl, pyrazolyl, indolyl, purinyl, carbazolyl, benzimidazolyl, and isoxazolyl. The optional substituent or substituents may be 1 to 3 members selected from the group consisting of C₁-C₆ alkyl, halogen, C₂-C₇ alkenyl, C₂-C₇ alkynyl, C₃-C₈ cycloalkyl, aralkyl, aryl optionally substituted with R₇, heterocycle optionally substituted with R, hydroxy, C₁-C₆ alkoxy, aryl-oxy, oxo (═O), —CN, —C(═O)H, —CO₂H, —OCO₂C₁-C₆ alkyl, —CO₂C₁-C₆ alkyl, —CO₂-aryl, —CO₂(C₁-C₆ alkyl)aryl, —OCO₂-aryl, —C(═O)NH₂, —C(═O)NHOH, amino, alkylamino, dialkylamino, —NHC(═O)NH—C₁-C₆ alkyl, —NHSO₂—C₁-C₆ alkyl, —NHSO₂-aryl, and —NHSO₂-heterocycle, where R is halo, C₁-C₆ alkoxy, C₁-C₆ alkyl, C₂-C₇ alkenyl, C₂-C₇ alkynyl, hydroxy, —C(═O)C₁-C₇ alkyl, —SO₂—C₁-C₆ alkyl, —CO₂—C₁-C₆ alkyl, C₂₋₇ acyl, or alkoxycarbonylalkyl.

The term “heteroarylmethyl,” as used herein, refers to the group R—CH₂— where R is a heteroaryl group, as defined herein.

The term “cycloalkyl,” as used herein, refers to an optionally substituted, alkyl group having one or more rings in their structures having from 3 to about 20 carbon atoms (and all combinations and subcombinations of ranges and specific numbers of carbon atoms therein), with from 3 to about 10 carbon atoms being preferred. Multi-ring structures may be bridged or fused ring structures. Groups include, but are not limited to, cyclopropyl, cyclobutyl, cyclopentyl, cyclohexyl, cyclooctyl, 2-[4-isopropyl-1-methyl-7-oxa-bicyclo[2.2.1]heptanyl], 2-[1,2,3,4-tetrahydro-naphthalenyl], and adamantyl. The optional substituent or substituents may be 1 to 3 members selected from the group consisting of C₁-C₆ alkyl, halogen, C₂-C₇ alkenyl, C₂-C₇ alkynyl, C₃-C₈ cycloalkyl, aralkyl, aryl optionally substituted with R₇, heterocycle optionally substituted with R, hydroxy, C₁-C₆ alkoxy, aryl-oxy, oxo (═O), —CN, —C(═O)H, —CO₂H, —OCO₂C₁-C₆ alkyl, —CO₂C₁-C₆ alkyl, —CO₂-aryl, —CO₂(C₁-C₆ alkyl)aryl, —OCO₂-aryl, —C(═O)NH₂, —C(═O)NHOH, amino, alkylamino, dialkylamino, —NHC(═O)NH—C₁-C₆ alkyl, —NHSO₂—C₁-C₆ alkyl, —NHSO 2-aryl, and —NHSO₂-heterocycle, where R is halo, C₁-C₆ alkoxy, C₁-C₆ alkyl, C₂-C₇ alkenyl, C₂-C₇ alkynyl, hydroxy, —C(═O)C₁-C₇ alkyl, —SO₂—C₁-C₆ alkyl, —CO₂—C₁-C₆ alkyl, C₂₋₇ acyl, or alkoxycarbonylalkyl.

The term “cycloalkylmethyl,” as used herein, refers to the group R—CH₂— where R is a cycloalkyl group, as defined herein.

The term “cycloalkenyl,” as used herein, refers to an optionally substituted, alkene group having one or more rings in their structures having from 3 to about 20 carbon atoms (and all combinations and subcombinations of ranges and specific numbers of carbon atoms therein), with from 3 to about 10 carbon atoms being preferred. Multi-ring structures may be bridged or fused ring structures. Groups include, but are not limited to, cyclopropenyl, cyclobutenyl, cyclopentenyl, cyclohexenyl, and cyclooctenyl.

The term “cycloalkenylmethyl,” as used herein, refers to the group R—CH₂— where R is a cycloalkenyl group, as defined herein.

The term “carbodiimide,” as used herein, refers to a compound of formula R—N═C═N—R, wherein each R is independently an optionally substituted cyclic or alicyclic aliphatic or aromatic hydrocarbon.

The term “sulfonamido,” or “sulfonamide,” as used herein, refers to a moiety containing the group —S(O)₂—NH—.

The term “sulfonyl,” or “sulfone,” as used herein, refers to a moiety containing the group —S(O)₂—.

The term “halo” or “halogen,” as used herein, refers to chloro, bromo, fluoro, and iodo.

As used herein, the term “contacting” refers to the bringing together of compounds to within distances that allow for intermolecular interactions and chemical transformations accompanying such interactions. Often, contacting compounds are in solution phase.

As used herein, the term “telescope,” in any verb form, refers to carrying out a series of steps in a chemical synthesis as sequential, one-pot syntheses that do not require separation and/or isolation steps between steps.

As used herein, the term “resolving” refers to any process of enhancing or enriching in a product the level of one enantiomer over its antipode from any mixture of the two enantiomers. Such mixtures include those where the enantiomers are present in equal amounts (racemates) or unequal amounts (those mixtures having an enantiomeric excess or one or the other of the enantiomers.

It is believed the chemical formulas and names used herein correctly and accurately reflect the underlying chemical compounds. However, the nature and value of the present invention does not depend upon the theoretical correctness of these formulae, in whole or in part. Thus it is understood that the formulas used herein, as well as the chemical names attributed to the correspondingly indicated compounds, are not intended to limit the invention in any way, including restricting it to any specific tautomeric form or to any specific optical or geometric isomer.

When ranges are used herein for physical properties, such as molecular weight, or chemical properties, such as chemical formulae, all combinations, and subcombinations of ranges specific embodiments therein are intended to be included.

When any variable occurs more than one time in any constituent or in any formula, its definition in each occurrence is independent of its definition at every other occurrence. Combinations of substituents and/or variables are permissible only if such combinations result in stable compounds.

In compounds where a carbon atom may be replaced by a heteroatom, such as a N, S, or O, each of such replacement groups may be substituted in the same manner as the carbon atom, if such substitution is technically feasible and does not violate valence or form an unstable species. Thus, for example, if any carbon ring atom may be substituted by —OH or R⁵, then the carbon atom (if replaced) may be NH, NR⁵, NOH, S, or O, even if such substitution is not explicitly stated.

Accordingly, in some embodiments, the present invention is directed to processes for preparing indolinone phenylaminopropanol compounds, comprising the steps of:

a. coupling a compound of formula IV:

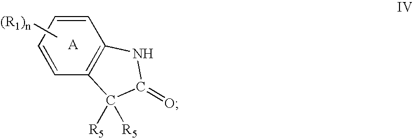

or a metal salt thereof;

with a compound of formula II:

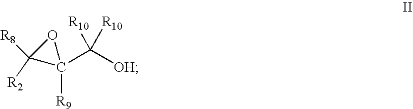

to form a diol compound of formula V:

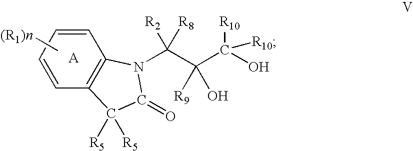

wherein said coupling is carried out in the presence of:
an optional Lewis acid catalyst;
a solvent composition comprising at least one polar, aprotic solvent; and
an excess of a strong non-nucleophilic base selected from the group consisting of RₓRₓ—N-M, R_{y}—O-M, and R_{y}—Mg—X;
where:
each Rₓ is independently alkyl substituted with 0-3 R₁, aryl substituted with 0-3 R₁, or (R_{z})₃Si;
or said Rₓ groups, together with the N atom to which they are attached, form a cyclic amine;
R_{y} is alkyl substituted with 0-3 R₁;
R_{z} is R₁;
M is Na, Li, or K;
X is Cl, Br, or I;
provided that said strong non-nucleophilic base is other than sodium t-butoxide;
wherein:
R₁ is, independently at each occurrence, alkyl, alkoxy, halo, CF₃, OCF₃, arylalkyloxy substituted with 0-3 R₁₁, aryloxy substituted with 0-3 R₁₁, aryl substituted with 0-3 R₁₁, heteroaryl substituted with 0-3 R₁₁, hydroxy, alkanoyloxy, nitro, nitrile, alkenyl, alkynyl, alkylsulfoxide, phenylsulfoxide substituted with 0-3 R₁₁, alkylsulfone, phenylsulfone substituted with 0-3 R₁₁, alkylsulfonamide, phenylsulfonamide substituted with 0-3 R₁₁, heteroaryloxy substituted with 0-3 R₁₁, heteroarylmethyloxy substituted with 0-3 R₁₁, alkylamido, or arylamido substituted with 0-3 R₁₁; or two adjacent R₁ also represent methylenedioxy;
R₂ is aryl substituted with 0-3 R₁ or heteroaryl substituted with 0-3 R₁;
R₅ is, independently at each occurrence, H, C₁-C₄ alkyl, aryl substituted with 0-3 R₁, or cyano; or the two R₅ form a carbocyclic ring of 3-7 carbons;
R₈ is H, or C₁-C₄ alkyl;
R₉ is H, or C₁-C₄ alkyl;
R₁₀ is, independently at each occurrence, H, or C₁-C₄ alkyl;
R₁₁ is alkyl, alkoxy, halo, CF₃, OCF₃, hydroxy, alkanoyloxy, nitro, nitrile, alkenyl, alkynyl, alkylsulfoxide, alkylsulfone, alkylsulfonamide, or alkylamido; or two adjacent R₁₁ also represent methylenedioxy;
n is an integer from 0 to 4; and
wherein 1-3 carbon atoms in ring A may optionally be replaced with N.

In certain preferred embodiments of this step of the process, the strong base is lithium hexamethyldisilazide (LHMDS). In certain preferred embodiments of this step of the process, the Lewis acid is titanium (IV) isopropoxide. The solvent composition comprising at least one polar, aprotic solvent is suitably a mixture comprising aprotic solvents in which at least one solvent is a polar, aprotic solvent. In certain preferred embodiments of this step of the process, the aprotic solvent composition comprises dimethylformamide (DMF). In certain preferred embodiments of this step of the process, the aprotic solvent composition further comprises tetrahydrofuran (THF) or toluene.

In certain embodiments, the compound of formula V may be purified by, for example, crystallization from solvents, such as toluene and heptane.

In certain embodiments, the processes further comprise the step of:
b. selectively activating the terminal hydroxy group of said diol compound of formula V with a compound of the formula (R₁₂SO₂)₂O or R₁₂SO₂Z with or without the use of a catalyst (such as dibutyltin or DMAP) in the presence of an optional base (such as triethylamine (TEA), N-methyl morpholine, N,N′-diisopropylethylamine (DIPEA), sodium carbonate (Na₂CO₃), potassium carbonate (K₂CO₃), or mixtures thereof in an inert solvent (such as tetrahydrofuran (THF), acetonitrile (CH₃CN), dichloromethane (CH₂Cl₂) or toluene) to form a compound of formula Va:

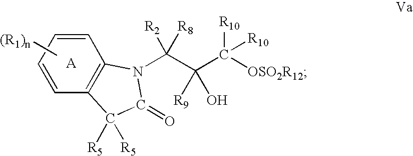

wherein:

Z is Cl or Br; and

R₁₂ is alkyl substituted with 0-3 R₁ or aryl substituted with 0-3 R₁. Preferably, the amount of catalyst used is about 0.1 mol % to about 100 mol %, preferably 1 to 5 mol % with temperature at about −20° C. to about 50° C. Preferably, the sulfonation is conducted with p-toluenesulfonyl chloride with triethylamine and catalytic dibutyl tin oxide in toluene, or acetonitrile, or mixture thereof.

Preferably, R₁₂ is methyl, ethyl, propyl, butyl, trifluoromethyl (triflate), phenyl, or benzyl, any of which may be optionally substituted with one or more substituents selected from the group consisting of C₁-C₄ alkyl, C₁-C₄ alkoxy, and halo (such as fluoro, chloro, and bromo). In certain preferred embodiments, R₁₂ is p-tolyl, methyl, brosyl, p-methoxyphenyl, p-ethoxyphenyl, pentafluorophenyl, or 2,4,6-triisopropyl.

In certain embodiments, the process further comprises the step of:
c. converting said compound of formula Va in the presence of a base and an optional phase transfer catalyst to a compound of formula VI:

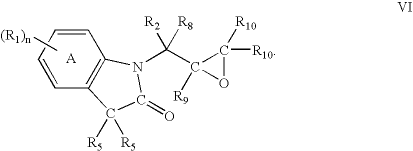

Preferably, the base is aqueous sodium hydroxide (NaOH), aqueous potassium hydroxide (KOH), aqueous potassium carbonate (K₂CO₃), or mixtures thereof. Sodium hydroxide is especially preferred. Preferably, the optional phase transfer catalyst is a compound of the formula (R₁₃)₄NX′, where:

R₁₃ is alkyl substituted with 0-3 R₁ or aryl substituted with 0-3 R₁; and

X′ is a counterion, such as Cl, Br, I, F, HSO₄, NO₃, OAc, OH, and the like. A preferred phase transfer catalyst is Bu₄NCl.

In alternate embodiments of forming compounds of formula VI, the processes further comprise the step of:
c. treating said compound of formula V with phosphine (such as triphenylphosphine) and dialkyl azodicarboxylate (such as diethyl azodicarboxylate or diisopropyl azodicarboxylate) in inert solvent (such as tetrahydrofuran (THF), toluene, or a mixture thereof) to form a compound of formula VI:

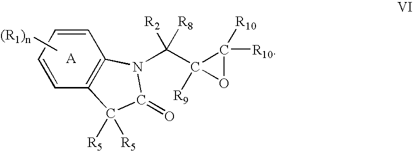

In certain embodiments, the processes further comprise the step of:
d. reacting said compound of formula VI with NHR₄R₄ with optional Lewis acid catalyst (such as Ca(OTf)₂, LiClO₄, or mixtures thereof) in an optional polar solvent (such as MTBE, methanol, ethanol, CH₃CN, water, or a mixture thereof) to form a compound of formula I:

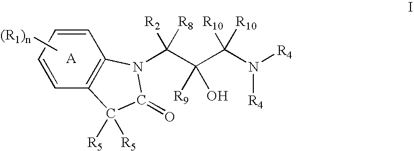

wherein:
R₄ is, independently at each occurrence, H, C₁-C₄ alkyl, arylalkyl, heteroarylmethyl, cycloheptylmethyl, cyclohexylmethyl, cyclopentylmethyl, or cyclobutylmethyl; and
with respect to the compound of formula I, R₁₀ and R₄, together with the nitrogen to which R₄ is attached, form a nitrogen-containing ring containing 3 to 6 carbons.
   Preferably, the reaction is carried out at about 10° C. to about 110° C. Preferably, the polar solvent is ethanol or methanol. Preferably, the Lewis acid catalyst is Ca(OTf)₂ at about 30° C. to about 45° C. The final free base of the compound of formula I may be optionally purified, for example, with acid/base extractions. Preferably, NHR₄R₄ is NH₂CH₃.

In certain embodiments, the processes further comprise the step of:
e. forming a pharmaceutically acceptable salt of said compound of formula I, especially a hydrochloride salt.

In certain preferred embodiments, steps b, c, and d are telescoped.

In certain embodiments, compound of formula II is formed from an allylic alcohol of formula III:

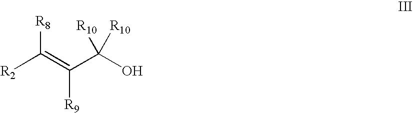

by reacting said compound of formula III with a homochiral diester of a tartaric acid (such as (-)-diisopropyl tartrate) and a hydroperoxide [such as t-butyl hydroperoxide (TBHP) or cumene hydroperoxide (CHP)], in the presence of a metal catalyst (including transition metal catalysts such as titanium (IV) isopropoxide) in optional inert solvent.

In certain preferred embodiments, the reaction of the compound of formula III is quenched with a reducing agent (such as sodium bisulfite) and optional citric acid.

In certain preferred embodiments, the allylic alcohol of formula III is formed by reducing a compound of formula VIII:

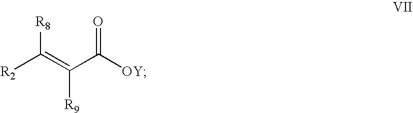

wherein:

Y is alkyl substituted with 0-3 R₁, aryl substituted with 0-3 R₁, or heteroaryl substituted with 0-3 R₁, preferably C₁-C₄ alkyl, and more preferably C₁ alkyl. Preferably, the alkyl ester of the compound of formula VII is reduced using a reducing agent, such as, for example, a hydride reagent, including DIBAL, Red-Al, L-selectride, K-selectride, and the like, in inert solvent, such tetrahydrofuran (THF) or toluene. The reaction is preferably quenched with a protic acid, such as hydrochloric acid, or a protic solvent such as ethanol.

In certain preferred embodiments, the compound of formula VII is formed by esterifying a compound of formula VIII:

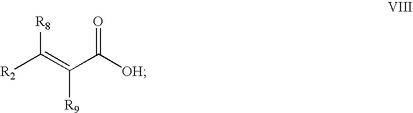

or a salt thereof;

The compound of formula VIII may be esterified (1) under standard acid-catalyzed conditions, such as p-toluenesulfonic acid (p-TSA) in an alkyl alcohol, such as methanol; or (2) under standard base-catalyzed conditions, such as CsCO₃, in alkyl halide, such as methyl iodide.

In certain embodiments, the steps of reducing the compound of formula VII and esterifying the compound of formula VIII may be telescoped.

In certain embodiments, the allylic alcohol of formula III may be isolated in a solution in an inert solvent.

In certain preferred embodiments, the compound of formula IV is formed from a compound of formula IX:

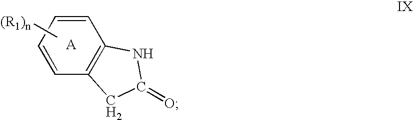

or a salt thereof.

In certain preferred embodiments, the compound of formula IX is formed by reducing a compound of formula X:

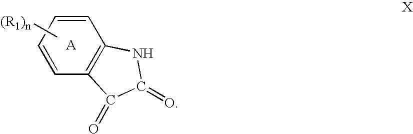

In certain embodiments, the compound of formula I is a compound of formula I*:

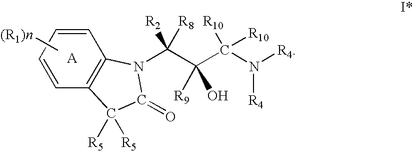

In certain preferred embodiments, the compound of formula I is

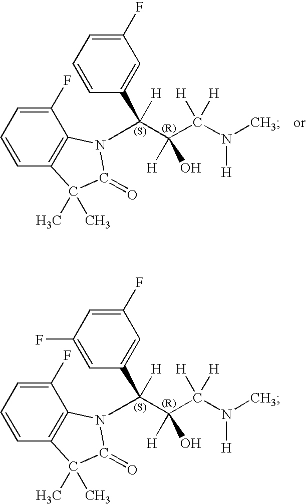

especially where the compound of formula I is

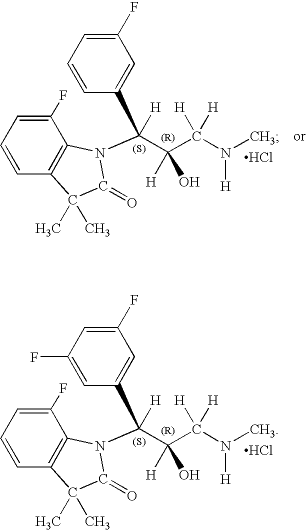

In certain embodiments, the compound of formula II is a compound of formula II*:

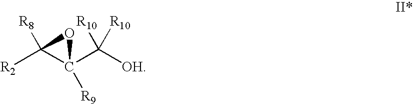

In certain embodiments, the compound of formula V is a compound of formula V*:

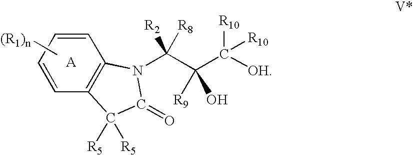

In certain embodiments, the compound of formula VI is a compound of formula VI*:

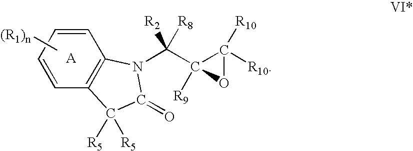

In other embodiments, the invention is directed to processes, comprising the step of:

aa. transesterifying a diol compound of formula V:

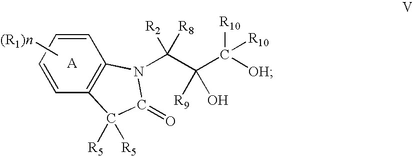

including a diol compound of formula V*

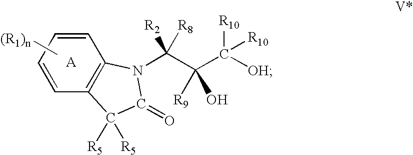

with a trialkyl orthoacetate (such as trimethyl orthoacetate) in the presence of a catalytic amount of an acid or an acid catalyst to form a cyclic orthoester compound of formula XI:

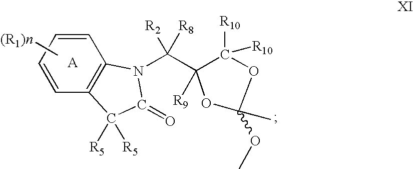

including a cyclic orthoester compound of formula XI*:

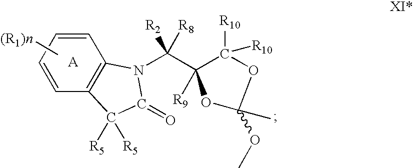

wherein:
R₁ is, independently at each occurrence, alkyl, alkoxy, halo, CF₃, OCF₃, arylalkyloxy substituted with 0-3 R₁₁, aryloxy substituted with 0-3 R₁₁, aryl substituted with 0-3 R₁₁, heteroaryl substituted with 0-3 R₁₁, hydroxy, alkanoyloxy, nitro, nitrile, alkenyl, alkynyl, alkylsulfoxide, phenylsulfoxide substituted with 0-3 R₁₁, alkylsulfone, phenylsulfone substituted with 0-3 R₁₁, alkylsulfonamide, phenylsulfonamide substituted with 0-3 R₁₁, heteroaryloxy substituted with 0-3 R₁₁, heteroarylmethyloxy substituted with 0-3 R₁₁, alkylamido, or arylamido substituted with 0-3 R₁₁; or two adjacent R₁ also represent methylenedioxy;
R₂ is aryl substituted with 0-3 R₁ or heteroaryl substituted with 0-3 R₁;
R₅ is, independently at each occurrence, H, C₁-C₄ alkyl, aryl substituted with 0-3 R₁, or cyano; or the two R₅ form a carbocyclic ring of 3-7 carbons;
R₈ is H, or C₁-C₄ alkyl;
R₉ is H, or C₁-C₄ alkyl;
R₁₀ is, independently at each occurrence, H, or C₁-C₄ alkyl;
R₁₁ is alkyl, alkoxy, halo, CF₃, OCF₃, hydroxy, alkanoyloxy, nitro, nitrile, alkenyl, alkynyl, alkylsulfoxide, alkylsulfone, alkylsulfonamide, or alkylamido; or two adjacent R₁₁ also represent methylenedioxy;
n is an integer from 0 to 4; and
wherein 1-3 carbon atoms in ring A may optionally be replaced with N.

In the alternative embodiment described immediately above, the process may further comprise the step of:
bb. reacting said cyclic orthoester compound of formula XI with a trimethylsilyl-X″ or acetyl-X″ to form a halohydrin ester of formula XII:

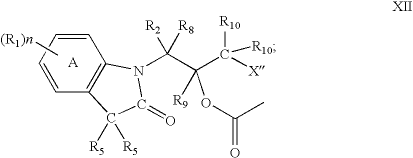

including a halohydrin ester of formula XII*

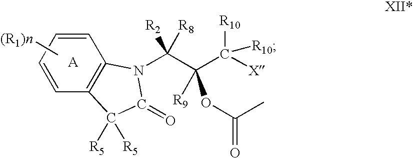

wherein:
   X″ is Cl, Br, or I.

In the alternative embodiment described immediately above, the process may further comprise the step of:
cc. treating said halohydrin ester of formula XII with base (such as potassium carbonate) in a polar solvent (such as ethanol) to cause ester saponification and cyclization form an epoxide compound of formula VI:

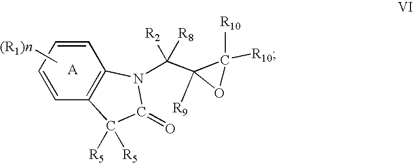

including an epoxide compound of formula VI*:

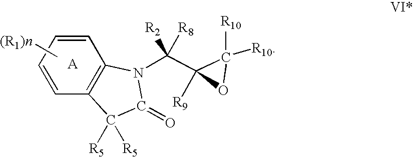

In the alternative embodiment described above with respect to step bb, processes may alternately further comprise the step of:
bb. converting said cyclic orthoester compound of formula XI to a halohydrin ester of formula XII:

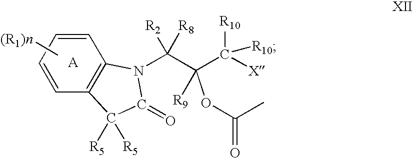

wherein:
   X″ is Cl, Br, or I.
and then converting said halohydrin ester compound of formula XII to a compound of formula VI:

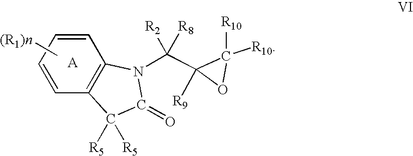

The cyclic orthoester compound of formula XI can be converted to the epoxide compound of formula VI via the formation of the halohydrin ester compound of formula XII. As a specific example,

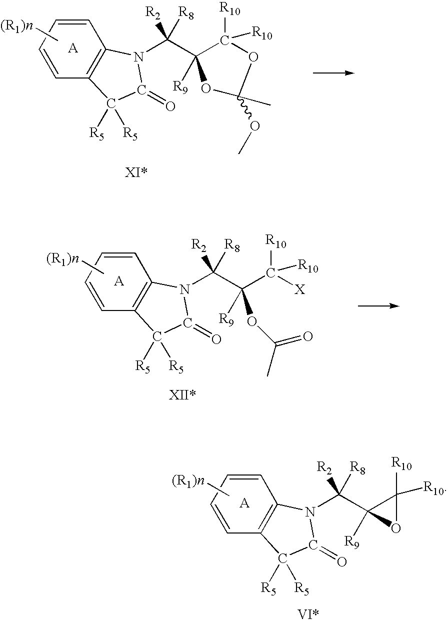

In the alternative embodiment described immediately above (from vicinal diol V via halohydrin ester XII to epoxide VI), the steps aa, bb, and cc are telescoped.

In the alternative embodiment described immediately above, the process may further comprise the step of:
dd. reacting said compound of formula VI with NHR₄R₄ and optional Lewis acid catalyst in an optional polar solvent to form a compound of formula I:

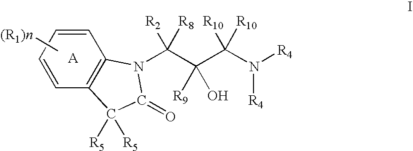

wherein:
R₄ is, independently at each occurrence, H, C₁-C₄ alkyl, arylalkyl, heteroarylmethyl, cycloheptylmethyl, cyclohexylmethyl, cyclopentylmethyl, or cyclobutylmethyl; and
with respect to the compound of formula I, R₁₀ and R₄, together with the nitrogen to which R₄ is attached, form a nitrogen-containing ring containing 3 to 6 carbons.

In the alternative embodiment described immediately above, step cc may be replaced with step ee to form compounds of formula I:

ee. reacting said halohydrin ester of formula XII:

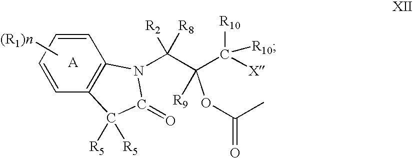

including a halohydrin ester of formula XII*

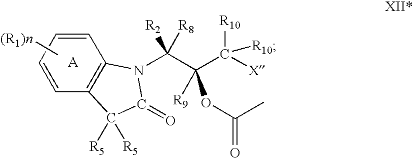

with NHR₄R₄ (such as a methyl alkyl amine) in an optional polar solvent (such as ethanol) to form a compound of formula I:

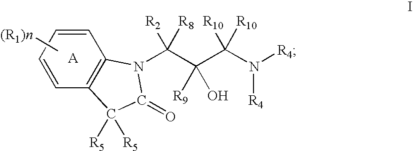

including a compound of formula I*:

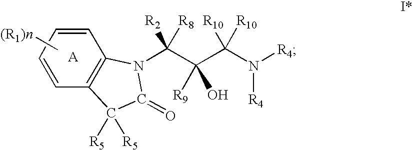

wherein:
R₄ is, independently at each occurrence, H, C₁-C₄ alkyl, arylalkyl, heteroarylmethyl, cycloheptylmethyl, cyclohexylmethyl, cyclopentylmethyl, or cyclobutylmethyl; and
with respect to the compound of formula I, R₁₀ and R₄, together with the nitrogen to which R₄ is attached, form a nitrogen-containing ring containing 3 to 6 carbons.

In other embodiments, the present invention is directed to an isolated, solid form of the intermediate compound of formula V:

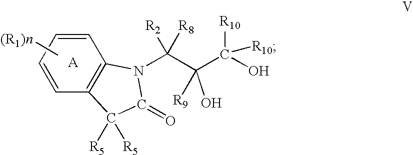

wherein:

R₁ is, independently at each occurrence, alkyl, alkoxy, halo, CF₃, OCF₃, arylalkyloxy substituted with 0-3 R₁₁, aryloxy substituted with 0-3 R₁₁, aryl substituted with 0-3 R₁₁, heteroaryl substituted with 0-3 R₁₁, hydroxy, alkanoyloxy, nitro, nitrile, alkenyl, alkynyl, alkylsulfoxide, phenylsulfoxide substituted with 0-3 R₁₁, alkylsulfone, phenylsulfone substituted with 0-3 R₁₁, alkylsulfonamide, phenylsulfonamide substituted with 0-3 R₁₁, heteroaryloxy substituted with 0-3 R₁₁, heteroarylmethyloxy substituted with 0-3 R₁₁, alkylamido, or arylamido substituted with 0-3 R₁₁; or two adjacent R₁ also represent methylenedioxy;

R₂ is aryl substituted with 0-3 R₁ or heteroaryl substituted with 0-3 R₁;

R₅ is, independently at each occurrence, H, C₁-C₄ alkyl, aryl substituted with 0-3 R₁, or cyano; or the two R₅ form a carbocyclic ring of 3-7 carbons;

R₈ is H, or C₁-C₄ alkyl;

R₉ is H, or C₁-C₄ alkyl;

R₁₀ is, independently at each occurrence, H, or C₁-C₄ alkyl;

R₁₁ is alkyl, alkoxy, halo, CF₃, OCF₃, hydroxy, alkanoyloxy, nitro, nitrile, alkenyl, alkynyl, alkylsulfoxide, alkylsulfone, alkylsulfonamide, or alkylamido; or two adjacent R₁₁ also represent methylenedioxy;

n is an integer from 0 to 4; and

wherein 1-3 carbon atoms in ring A may optionally be replaced with N.

In yet other embodiments, the present invention is directed to intermediate compounds of formula VI:

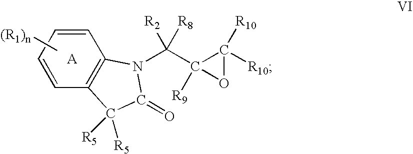

or a pharmaceutically acceptable salt thereof;

wherein:

R₁ is, independently at each occurrence, alkyl, alkoxy, halo, CF₃, OCF₃, arylalkyloxy substituted with 0-3 R₁₁, aryloxy substituted with 0-3 R₁₁, aryl substituted with 0-3 R₁₁, heteroaryl substituted with 0-3 R₁₁, hydroxy, alkanoyloxy, nitro, nitrile, alkenyl, alkynyl, alkylsulfoxide, phenylsulfoxide substituted with 0-3 R₁₁, alkylsulfone, phenylsulfone substituted with 0-3 R₁₁, alkylsulfonamide, phenylsulfonamide substituted with 0-3 R₁₁, heteroaryloxy substituted with 0-3 R₁₁, heteroarylmethyloxy substituted with 0-3 R₁₁, alkylamido, or arylamido substituted with 0-3 R₁₁; or two adjacent R₁ also represent methylenedioxy;

R₂ is aryl substituted with 0-3 R₁ or heteroaryl substituted with 0-3 R₁;

R₅ is, independently at each occurrence, H, C₁-C₄ alkyl, aryl substituted with 0-3 R₁, or cyano; or the two R₅ form a carbocyclic ring of 3-7 carbons;

R₈ is H, or C₁-C₄ alkyl;

R₉ is H, or C₁-C₄ alkyl;

R₁₀ is, independently at each occurrence, H, or C₁-C₄ alkyl;

R₁₁ is alkyl, alkoxy, halo, CF₃, OCF₃, hydroxy, alkanoyloxy, nitro, nitrile, alkenyl, alkynyl, alkylsulfoxide, alkylsulfone, alkylsulfonamide, or alkylamido; or two adjacent R₁₁ also represent methylenedioxy;

n is an integer from 0 to 4; and

wherein 1-3 carbon atoms in ring A may optionally be replaced with N.

In another embodiment, the present invention is directed to the products produced by the above-described processes. These products are mixtures that include lower levels of impurities than the prior art products.

In another embodiment, the invention is directed to compositions having reduced levels of the dehydration impurity, comprising:

a compound of formula I; and

less than about 35%, preferably less than about 25%, even more preferably less than about 20%, yet even more preferably less than about 10%, and more preferably less than about 5% by weight, based on the total weight of the composition, of a compound of formula I′:

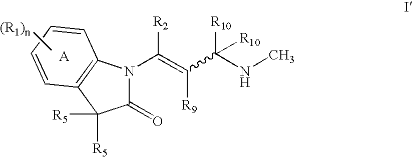

R₁ is, independently at each occurrence, alkyl, alkoxy, halo, CF₃, OCF₃, arylalkyloxy substituted with 0-3 R₁₁, aryloxy substituted with 0-3 R₁₁, aryl substituted with 0-3 R₁₁, heteroaryl substituted with 0-3 R₁₁, hydroxy, alkanoyloxy, nitro, nitrile, alkenyl, alkynyl, alkylsulfoxide, phenylsulfoxide substituted with 0-3 R₁₁, alkylsulfone, phenylsulfone substituted with 0-3 R₁₁, alkylsulfonamide, phenylsulfonamide substituted with 0-3 R₁₁, heteroaryloxy substituted with 0-3 R₁₁, heteroarylmethyloxy substituted with 0-3 R₁₁, alkylamido, or arylamido substituted with 0-3 R₁₁; or two adjacent R₁ also represent methylenedioxy;

R₂ is aryl substituted with 0-3 R₁ or heteroaryl substituted with 0-3 R₁;

R₅ is, independently at each occurrence, H, C₁-C₄ alkyl, aryl substituted with 0-3 R₁, or cyano; or the two R₅ form a carbocyclic ring of 3-7 carbons;

R₉ is H, or C₁-C₄ alkyl;

R₁₀ is, independently at each occurrence, H, or C₁-C₄ alkyl;

R₁₁ is alkyl, alkoxy, halo, CF₃, OCF₃, hydroxy, alkanoyloxy, nitro, nitrile, alkenyl, alkynyl, alkylsulfoxide, alkylsulfone, alkylsulfonamide, or alkylamido; or two adjacent R₁₁ also represent methylenedioxy;

n is an integer from 0 to 4;

wavy line represents both stereochemical configurations between the carbons to which R₉ and R₁₀ are attached; and

wherein 1-3 carbon atoms in ring A may optionally be replaced with N. The two examples of the stereoisomers of the compounds of formula I′ are:

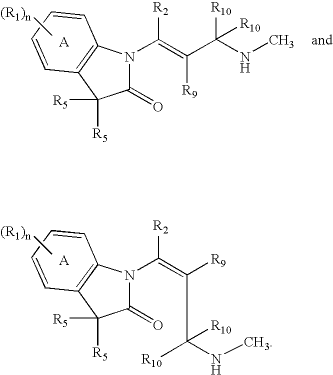

In certain preferred embodiments of the above-described processes, compounds, and compositions, R₁ is, independently at each occurrence, halo, especially F.

In certain preferred embodiments of the above-described processes, compounds, and compositions, R₂ is aryl substituted with R₁, especially R₂ is phenyl substituted with one or more F, and more especially, R₂ is m-fluorophenyl or 3,5-difluorophenyl.

In certain preferred embodiments of the above-described processes, compounds, and compositions, R₄ is, independently at each occurrence, H or C₁ alkyl.

In certain preferred embodiments of the above-described processes, compounds, and compositions, R₅ is C₁ alkyl.

In certain preferred embodiments of the above-described processes, compounds, and compositions, R₈ is H.

In certain preferred embodiments of the above-described processes, compounds, and compositions, R₉ is H.

In certain preferred embodiments of the above-described processes, compounds, and compositions, R₁₀ is H.

In certain preferred embodiments of the above-described processes, compounds, and compositions, n is 1.

### General Procedure

One method of preparing the compounds of formula I is shown in Scheme A, using 7-fluoro-1-[(1S,2R)-1-(3-fluorophenyl)-2-hydroxy-3-(methyl amino)propyl]-3,3-dimethyl-1,3-dihydro-2H-indol-2-one as a representative example. The synthesis begins from 3-fluorocinnamic acid. 3-Fluorocinnamic acid is esterified under standard acid catalyzed esterification conditions with acid such as p-TSA in an alkyl alcohol such as methyl alcohol or base catalyzed esterification conditions with base, such as CsCO₃, in alkyl halide, such as methyl iodide (MeI).

The resulting alkyl ester is reduced using a hydride reagent such as DIBAL, Red-Al, L-selectride, K-selectride, and the like in an inert solvent such as THF or toluene. Preferably, the reduction was conducted with DIBAL in toluene. The reaction was worked up by quenching into a protic acid (HCl) or a protic solvent.

The esterification and reduction steps can be telescoped and the allylic alcohol can be isolated as a toluene solution.

The allylic alcohol can then be diastereoselectively epoxidized to give the (R, R) epoxy alcohol. For example, the epoxidation can be accompanied using a homochiral diester of a tartaric acid, a hydroperoxide, and a metal catalyst, such as a transition metal catalyst. In one embodiment, the homochiral diester is (-)-diisopropyl tartrate ((-)-DIPT), the hydroperoxide is t-butyl hydroperoxide (TBHP) or cumene hydroperoxide (CHP), and the metal catalyst is titanium (IV) isopropoxide. Preferably, the reaction is carried out in an inert solvent such as toluene or dichloromethane. The amount of catalyst used is 2-100 mol %, preferably 5-10 mol % with the temperature at −60° C. to −20° C., preferably at −35° C. to −20° C. The reaction is quenched with a reducing agent such as sodium bisulfite or Fe₂SO₄ with or without the use of citric acid.

The epoxide can be subsequently coupled to an alkaline metal salt of a dimethyl oxindole with bases, such as LiHMDS, KHMDS, LDA, or KOtBu with or without the use of a transition metal catalyst, such as Ti(iPrO)₄, in an aprotic solvent such as THF, toluene, DMF or mixture thereof. Preferably, the coupling is conducted with LiHMDS as base and with Ti(iPrO)₄ as a transition metal catalyst. The diol can be purified by crystallization from solvents, such as toluene and heptane.

The primary hydroxyl of the diol can be activated as a sulfonate; such as p-toluenesulfonate, methanesulfonate, triisopropylsulfonate, or 2,4,6-trimethylbenzene-sulfonate, with or without catalyst, such as dibutyltin oxide or DMAP with base, such as TEA, N-methyl morpholine, DIPEA, Na₂CO₃, or K₂CO₃ in an inert solvent, such as CH₃CN, CH₂Cl₂, or toluene. Preferably, the sulfonation is conducted with p-toluenesulfonyl chloride with TEA and catalytic dibutyltin oxide in toluene. The amount of catalyst used is 0.1-100 mol %, preferably 1-5 mol % with the temperature at −20 to 50° C., preferably at −5 to 5° C.

The sulfonate can be treated with a base, such as NaOH, KOH, K₂CO₃ and the like, to give the epoxide. Preferably, the base is NaOH. In certain embodiments, tosylate may also be displaced by methylamine and form final amine.

Alternatively, the epoxide can be formed under Mitsunobu condition. Diol can be treated with phosphine, such as triphenylphosphine, and dialkyl azodicarboxylate, such as diethyl dicarboxylate, in an inert solvent, such as THF or toluene.

Alternatively, the epoxide can be formed from the cyclic orthoester to halohydrin ester, as shown in Scheme B.

The epoxide can be opened by methylamine with or without Lewis acid catalyst such as Ca(OTf)₂, LiClO4 in polar solvent such as MTBE, MeOH, EtOH, CH₃CN, H₂O or mixture thereof at 10 to 110° C. Preferably, the amine opening is conducted in EtOH with Ca(OTf)₂ as a catalyst at 30 to 45° C. The final free base can be purified from acid/base extractions.

Furthermore, the sulfonation, base catalyzed epoxide formation, epoxide opening by methylamine, and salt formation can be telescoped.

Other salts can be formed with the final free base.

Scheme C shows the formation of the diol and epoxide intermediates that may be used in the formation of 1-[1-(3,5-difluoro-phenyl)-2-hydroxy-3-methylamino-propyl]-7-fluoro-3,3-dimethyl-1,3-dihydro-indol-2-one.

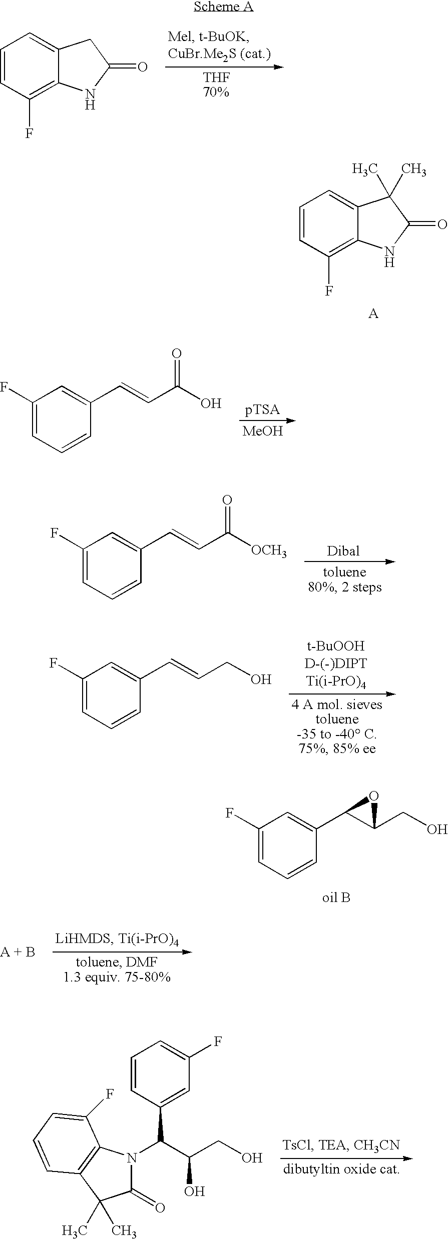

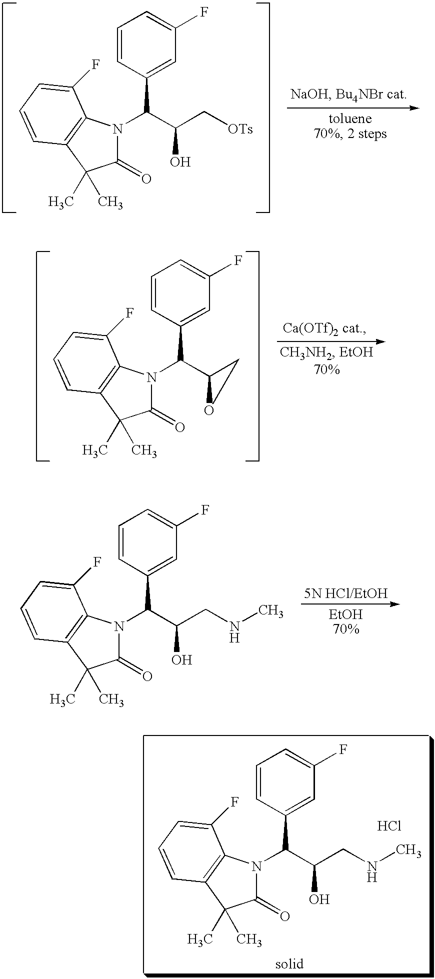

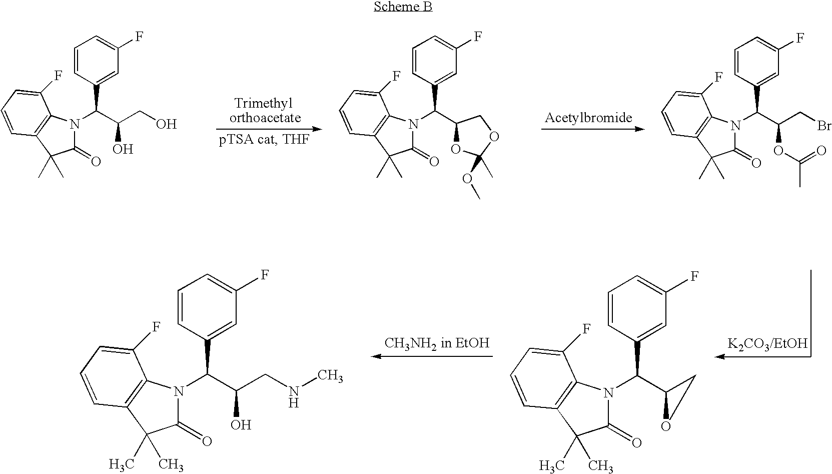

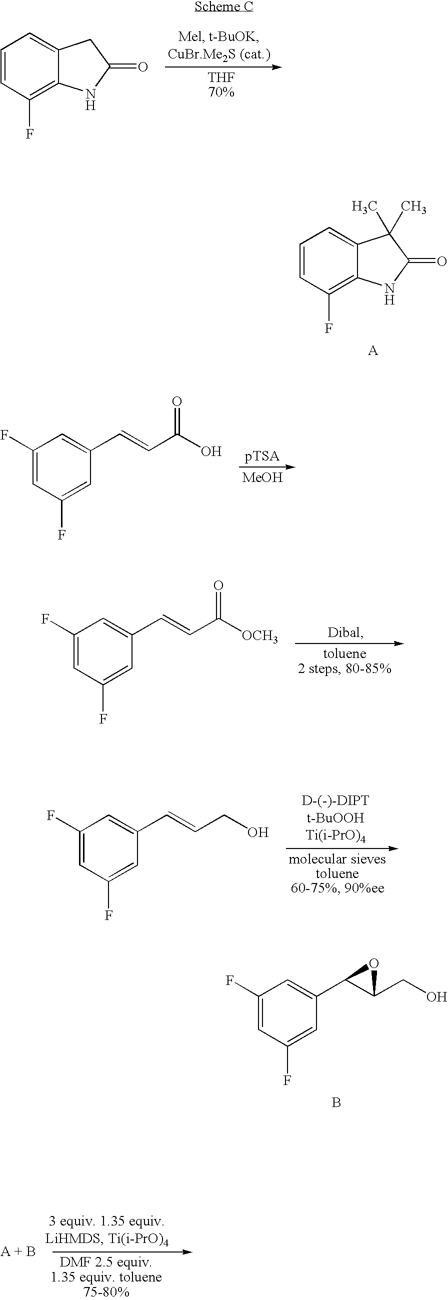

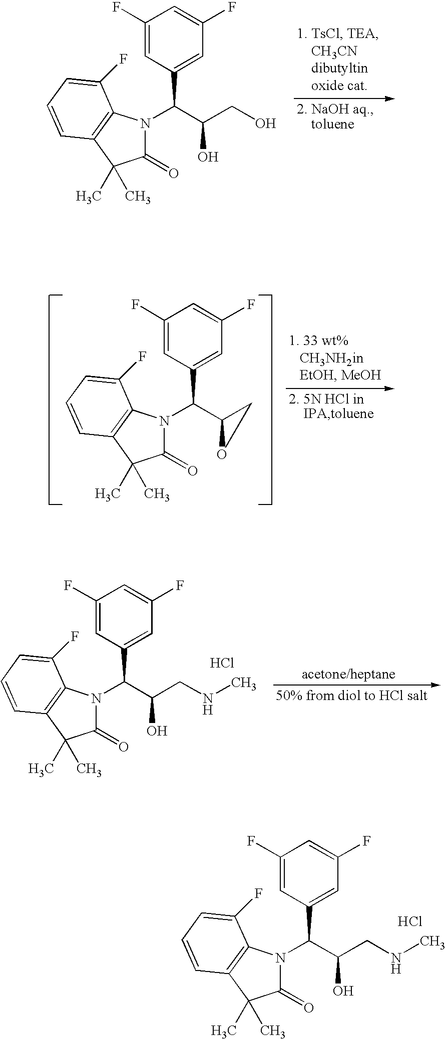

The present invention is further defined in the following Examples, in which all parts and percentages are by weight and degrees are Celsius, unless otherwise stated. It should be understood that these examples, while indicating preferred embodiments of the invention, are given by way of illustration only. From the above discussion and these examples, one skilled in the art can ascertain the essential characteristics of this invention, and without departing from the spirit and scope thereof, can make various changes and modifications of the invention to adapt it to various usages and conditions.

### EXAMPLES

### Analytical

NMR spectra of the intermediates were recorded on a Bruker Avance DPX 300 NMR spectrometer. Spectra were referenced by an internal standard.

HPLC analysis of the intermediates and reaction monitoring was carried out on an Agilent 1090 liquid chromatograph equipped with a Phenomenex Prodigy ODS3 4.6×50 mm column. Standard method: 90:10 to 10:90 over 8 minutes gradient of water-acetonitrile containing 0.02% TFA, flow rate 1 ml/min.

LCMS data were obtained on an Agilent 1100 LC system with an Agilent 1100 LC/MS detector equipped with a 4.6×50 mm Chromolith SpeedROD column. Standard method: 90:10 to 10:90 over 4 minutes gradient of water-acetonitrile containing 0.02% TFA, flow rate 4 ml/min.

Analytical instrumentation and methods used for the analysis of the final material are described below together with the analytical data.

All starting materials are commercially available, unless otherwise noted.

### Example 1

### Preparation of (1R,3R)-3-(3-fluorophenyl)-2-(hydroxymethyl)oxirane

A thoroughly dried 5-L jacketed reactor was equipped with a mechanical stirrer, a 500-mL addition funnel, a temperature probe and a nitrogen inlet. The reactor was charged with D-(-)-DIPT (13.0 g 46 mmol), 4-Å 5-μm molecular sieves (90 g) and dichloromethane (4.00 L) and then it was purged with nitrogen. The contents of the reactor were cooled to −15° C. Titanium isopropoxide (12.19 g, 43 mmol) was added rapidly to the reaction mixture via the addition funnel and the reaction mixture was further cooled to −20° C. A solution of allylic alcohol (127 g, 0.854 mol) in CH₂Cl₂ (380 mL) was added to the reaction mixture via the addition funnel at a rate to keep the temperature in the reactor below −20° C. The resulting mixture was allowed to stir at −20° C. for 10 minutes. A solution of TBHP in CH₂Cl₂ (4.5 M, 380 mL, 1.71 mol) was added to the reaction mixture via the addition funnel at a rate to maintain the temperature below −20° C. and above −25° C. (addition rate 7 ml/min). The reaction mixture was stirred at −20° C. for 4 hours. Reaction progress was monitored by HPLC: an aliquot was drawn out of the reactor and diluted with MeCN-water. The reaction was deemed complete when the amount of the starting olefin fell below 1%.

The reaction mixture was transferred from the reactor into a 6-L flask containing a solution of FeSO₄×7H₂O (356 g, 1.28 mol), citric acid monohydrate (93 g, 0.39 mol) and de-ionized water (to the total volume of 1.0 L) chilled in an ice bath to 0° C. The rate of transfer was adjusted to maintain the temperature of the mixture below 10° C. The flask with the resulting mixture was equipped with a mechanical stirrer and the mixture was stirred for 25 minutes.

The organic layer was separated and filtered through a pad of Celite. The aqueous phase was extracted with MTBE (2×300 mL). Combined organic solutions were cooled to 0° C. in an ice bath. A 30% solution of NaOH (100 mL) in brine (prepared by dissolving 5 g of NaCl in a solution of NaOH (30.0 g) in 90 mL of water) was cooled in an ice bath to 0° C. and then added to the combined organic phases. The resulting mixture was stirred rapidly for 2 hours at 0° C. Water (400 mL) was added to the mixture and the layers were separated. The aqueous layer was extracted with MTBE (2×250 mL). The combined organic layers were dried with Na₂SO₄ (300 g), the drying agent was filtered off through a paper filter and the filtrate was evaporated on rotary evaporator. The oily residue was mixed with 700 mL of toluene and the solvent was removed on a rotary evaporator.

The residue after evaporation: Weight 125.9 g.

HPLC purity (area % 215 nm): 94%, impurities: toluene (3.1%), starting olefin (1.0%), 3 unknown impurities (<0.7% each).

¹H NMR (CDCl₃). Impurities: toluene (1.7 weight %), DIPT (1.1 weight %), t-BuOH (0.4 weight %).

### Example 2

### Preparation of 7-fluoro-1-[(1S,2S)-1-(3-fluorophenyl)-2,3-dihydroxypropyl]-3,3-dimethylindolin-2-one

7-Fluoro-3,3-dimethyl-1,3-dihydro-2H-indol-2-one (60 g, 335 mmol) was mixed under nitrogen with anhydrous dimethylformamide (DMF) (10.8 mL). To the resulting viscous solution, cooled to 5-7° C., was added via syringe a solution of LiHMDS in THF (1M in THF, 140 ml, 140 mmol) at a rate to keep the reaction mixture temperature below 7-10° C. (addition of the first 60 ml was very exothermic, later the rate of addition could be increased). The resulting purple-red clear solution was allowed to warm up to 10° C.

In a separate flask, [(2R,3R)-3-(3-fluorophenyl)oxiran-2-yl]methanol (59.1 g, 352 mmol, 1.05 eq.) was dissolved in 600 mL of anhydrous THF, the flask was purged with nitrogen and the solution was cooled to 5-7° C. Titanium isopropoxide (104 ml, 100 g, 584 mmol) was added to the epoxide solution dropwise via syringe maintaining the temperature in the 7-12° C. range. The resulting bright-yellow solution was stirred for 40 minutes, allowing it to warm up to room temperature.

The contents of the second flask, the epoxy-titanium solution, were transferred to the solution of the indolinone salt via cannula maintaining the temperature of the mixture below 15° C. The resulting mixture was stirred at room temperature. The reaction progress was monitored by HPLC: after 20 hours, about 17 area % of indolinone was left, while no epoxide was detectable. Additional amount of the epoxide-titanium isopropoxide complex was prepared from epoxide (9.85 g, 58.4 mmol) and titanium isopropoxide (17.3 ml, 16.6 g, 58.4 mmol) in THF (100 mL) as described above and added slowly to the reaction mixture. The mixture was kept at room temperature for 20 hours longer, at which point HPLC analysis showed 4 area % of the unreacted indolinone and no detectable amount of the epoxide.

The reaction mixture was transferred into 1.80 L of cold (0° C.) 2 M aqueous HCl solution (Exotherm. The rate of addition was adjusted to keep the temperature below 15° C.). The resulting clear solution was extracted with MTBE (3×800 ml), the combined organic phase were washed with brine (800 ml), dried over magnesium sulfate and filtered through a pad of magnesol. The filtrate was evaporated, diluted with toluene (600 ml), and evaporated again to remove maximum amount of solvents. The residue (133 g) contained a sufficiently pure product to be used in the next step without further purification.

HPLC purity (area % at 215 nm): 95%, impurities: indolinone (3.5%).

¹H NMR (CDCl₃). Impurities: residual solvents (DMF, toluene, MTBE).

### Example 3

### Preparation of 7-fluoro-1-((S)-(3-fluorophenyl)((S)-oxiran-2-yl)methyl)-3,3-dimethylindolin-2-one

A 2-L round bottom flask, equipped with a mechanical stirrer, a 100-mL addition funnel, a temperature probe and a nitrogen inlet, was charged with a solution of 7-fluoro-1-[(1S,2S)-1-(3-fluorophenyl)-2,3-dihydroxypropyl]-3,3-dimethylindolin-2-one (50.0 g, 144 mmol) in CH₂Cl₂ (500 mL), triethylamine (62 mL, 0.433 mol), solid dibutyltin oxide (716 mg, 2.9 mmol) and DMAP (1.74 g, 14.4 mmol). Tosyl chloride (28.23 g, 148 mmol) was dissolved in CH₂Cl₂ (60 mL) and the solution was added slowly to the reaction mixture (addition rate 5.6 mL/min). Temperature range 20° C. to 23° C. The reaction flask was cooled in an ice water bath during the addition to keep the temperature below 25° C. After the addition was finished, the bath was removed and the reaction mixture was stirred at room temperature. The reaction progress was monitored by HPLC.

After about one hour, the amount of the diol fell below 10%. A solution of NaOH, prepared by diluting 72 mL of 10 M aqueous NaOH with 360 mL of deionized water, was added rapidly to the reaction mixture. Solid Bu₄N⁺C⁻ hydrate (2.05 g, 7.2 mmol) was added and the reaction mixture was stirred rapidly at room temperature. The progress of the epoxide closure was monitored by HPLC. After 2 hour, all tosylate was consumed.

The layers were separated. The aqueous layer was extracted with 100 mL of CH₂Cl₂. Combined organic solutions were washed with 100 mL portions of 0.5 M aqueous HCl until pH of the washes fell below 5, then with 50 mL of 0.5 M aqueous NaOH, then it was dried with Na₂SO₄. The solution was gravity-filtered through a pad of Silica gel (150 g, thickness of the pad 5 cm) prepared in a glass filter funnel. The drying agent and the pad were washed with dichloromethane. The washing continued until no more epoxide was detectable in the eluent (by HPLC). The filtrate was evaporated to dryness on rotary evaporator (room temp. bath).

The residue after evaporation: weight 42.6 g. HPLC purity 82%, impurities: bis-tosylate (12%), diol (2.5%), indolinone (2.4%). The crude intermediate was used in the next step without further purification.

### Example 4

### Preparation of 7-fluoro-1-[(1S,2R)-1-(3-fluorophenyl)-2-hydroxy-3-(methyl amino)propyl]-3,3-dimethylindolin-2-one hydrochloride

The residue from the previous step (42.6 g) was dissolved in ethanol (160 mL) and the solution was placed into a 1-L round bottom flask equipped with a mechanical stirrer and a temperature probe. Aqueous methylamine (40 weight %, 240 mL, 2.74 mol) was added to the solution and the resulting suspension was stirred at room temperature. The reaction was monitored by HPLC. After 15 hours, the amount of the epoxide fell below 1%. Ethanol was removed on rotary evaporator (bath temperature 27° C.). The residue was mixed with MTBE (250 mL) and water (100 mL). The layers were separated. The aqueous layer was extracted with 50 mL of MTBE. Combined organic solutions were washed with 100 mL of water. Small amount of brine was added to speed up the phase separation. The resulting organic solution was extracted with aqueous HCl (200 mL of 2 M solution, then 50 mL of 1 M solution). Combined acidic extracts were washed with 50 mL of MTBE.

MTBE (200 mL) was added to the aqueous solution. Aqueous NaOH (10 M solution, 50 mL, 500 mmol) was added to the bi-phasic mixture. The mixture was shaken and the layers were separated. The aqueous layer was extracted with MTBE (100 mL). Combined organic solutions were dried with Na₂SO₄ (75 g). The drying agent was filtered off and the filtrate was evaporated in vacuum.

The residue (38.0 g) was mixed with 70 mL of ethanol and the solvent was removed on rotary evaporator. The residue was re-dissolved in 100 mL of ethanol. With magnetic stirring, 2 M HCl in Et₂O (57 mL, 114 mmol) was added to the solution. The acidity of the solution was checked by placing a drop of the solution on a wet pH paper to ensure the solution is strongly acidic. The resulting solution was seeded with crystals of 7-fluoro-1-[(1S,2R)-1-(3-fluorophenyl)-2-hydroxy-3-(methyl amino)propyl]-3,3-dimethyl-1,3-dihydro-2H-indol-2-one hydrochloride salt which caused slow crystallization of the salt in about 30 minutes. The slurry was stirred at room temperature for 1 hour.

The reaction flask was placed into a 0° C. bath (equipped with thermostat) and the slurry was stirred magnetically for 21 hours. The cold slurry was filtered through a paper filter. The solid was washed with a 1:1 mixture of EtOH-Et₂O (70 mL) and then was dried on the filter in a stream of air for 2 hours.

Weight of the crystals 29.7 g (54% from theoretical yield calculated from the diol).

HPLC purity (area % at 215 nm): 98.2%, impurities (relative retention time): 1.05 (0.46%), 0.98 (0.42%), 1.07 (0.15%), 2.05 (0.14%).

Enantiomeric purity 99.4% ee.

m.p. 209.5-211.2° C.

[α]_{D}^{25°}=+10.70.

¹H NMR (D₂O, 400 MHz), δ: 7.45-7.30 (m, 3H), 7.16-6.97 (m, 4H), 5.53-5.30 (2H, broad m), 3.35-3.24 (2H, broad m), 2.82 (s, 3H), 1.41 (s, 3H), 1.27 (broad s, 3H).

Impurities: ethanol (0.3 weight %).

ES⁺MS, m/z 361 (MH⁺).

Anal. calc'd for C₂₀H₂₃ClF₂N₂O₂ (396.9): C, 60.53; H, 5.84; N, 7.06. Found: C, 60.43; H, 5.69; N, 6.84.

Sn content: 3 ppm.

### Example 5

### Preparation of 7-fluoro-3,3-dimethyl-oxindole via selective C-methylation of 7-fluoro-oxindole

To a stirred slurry of potassium tert-butoxide (185 g, 1.65 mol) in tetrahydrofuran (1350 mL) was added 7-fluoro-oxindole (50 g, 0.33 mol) and copper (I) bromide-dimethyl sulfide complex (7 g, 0.033 mol). Methyl iodide (150 g, 1.06 mol.) was added to the mixture at 5-10° C. The reaction mixture was stirred at 20-25° C. for 1 hour. 10% NH₄Cl (1000 mL) was added to the reaction mixture. The two layers were separated. The organic layer was concentrated via vacuum distillation at 25-40° C. to reach a volume of 250 mL. The aqueous layer is extracted with tert-butyl methyl ether (2×500 mL). The concentrated organic layer and tert-butyl methyl ether extraction layers were combined and washed with 15% NaCl (250 mL). The organic solution was filtered through silica gel (100 g). Heptane (1250 mL) was added to the filtrate. The mixture was concentrated under atmosphere at 60-95° C. to reach a volume of 700 mL. The concentrate was cooled to 0-5° C. from 85-95° C. over 2 hours to crystallize. Solid was filtered, washed with heptane (100 mL), and oven-dried to give 41 g (69.4%) of a beige solid 7-fluoro-3,3-dimethyl-oxindole, 92% w/w purity by HPLC.

### Example 6

### Preparation of 3-(3-fluoro-phenyl)-prop-2-en-1-ol

A 5-L reactor equipped with a mechanical stirrer, thermocouple, and nitrogen inlet was charged with MeOH (1.40 L) and 3-fluorocinnamic acid (0.20 kg, 1.20 mol). To the slurry charged p-TSA (0.023 kg, 0.120 mol) at 20° C. to 25° C. The suspension was refluxed at 65° C. to 68° C. for 3-5 hours. The mixture was concentrated via atmospheric distillation to reach a volume of 700 mL. Methanol was then chased off by adding toluene (1.8 L) and was further concentrated to a solution (about 1.5 L). The reaction mixture then washed successively with 5% aqueous NaHCO₃ (1.5) and water (1.5 L). The organic mixture was concentrate via atmospheric distillation to a minimum volume of 500 mL. HPLC analysis indicates that the solution strength 53.5% KF 0.17%, 98.8% area HPLC purity of the product.

A 3-L reactor equipped with a mechanical stirrer, thermocouple, and nitrogen inlet was charged with diisobutylaluminum hydride 25% w/w in Toluene (1.56 kg, 1.85 L, 2.75 mol). The solution was cooled to −25° C. To the reactor was then added using FMI pump a solution of 3-(3-Fluoro-phenyl)-acrylic acid methyl ester (0.41 kg, 0.40 L, 1.20 mol) in toluene while maintaining the internal temperature between −15° C. to −8° C. The reaction mixture was stirred at −15 to −8° C. for 60 minutes. The reaction mixture was then quenched in a 5-L reactor into a solution of concentrated HCl (0.40 L, 0.48 kg; 4.87 mol) in water (0.75 kg) maintaining internal temperature at 40° C. to 45° C. The biphasic mixture was separated. The lower aqueous layer was washed with Toluene (0.34 kg, 0.40 L). The combined organic phase was successively washed with a 5% aqueous solution of sodium bicarbonate (0.7 L) and 10% brine (0.7 L). The organic solution was concentrated via atmospheric distillation to reach a volume of 500 mL. HPLC analysis indicates that the solution strength is 53%, 169 g (93% Y), Al: 9 ppm, KF: 0.04%, 99% area HPLC purity of the allylic alcohol.

### Example 7

### Preparation of [3-(3-fluoro-phenyl)-oxiranyl]-methanol

A 3-L reactor equipped with a mechanical stirrer, thermocouple, and nitrogen inlet was charged with toluene (200 mL) and pre-activated molecular sieves powder (4A, 70 g). The resultant slurry was cooled to −35° C. To the reactor was then added a solution of D-(-)-diisopropyl tartrate (21.6 g, 92.0 mmol) in toluene (25 mL), followed by addition of titanium (IV) isopropoxide (18.7 g, 65.7 mmol). The temperature of the reaction mixture was maintained between −30° C. to −40° C. during the addition. To the reactor was then charged with a solution of 3-(3-fluoro-phenyl)-prop-2-en-1-ol (100 g, 657 mmol) in toluene (490 mL) while maintaining the temperature of the reaction mixture between −30° C. to −40° C. The reaction mixture was stirred at −35° C. for 30 minutes. To the reactor was then added a solution of 5.5 M tert-butyl hydroperoxide in decane (240 mL, 1310 mmol) while maintaining the temperature of the reaction mixture between −30° C. to −40° C. The reaction mixture was stirred at −35° C. for 6 hours, followed by 8 hours at −20° C. The reaction mixture was warmed to room temperature and filtered through a thin layer of celite. The filter cake was washed with toluene (2×100 mL). The combined filtrate and washes were cooled to 0° C. and a solution of 30% sodium hydroxide saturated with sodium chloride (100 mL) was then added. The reaction mixture is stirred at 0° C. for 2 hours. To the reaction mixture was then added a solution of sodium metabisulfite (69 g) and citric acid (50 g) in water (600 mL). The biphasic mixture was stirred at room temperature for 1 hour and the phases were separated. The organic phase was successively washed with a 5% sodium bicarbonate (500 mL) and 10% brine (500 mL). The organic solution was then concentrated under vacuum to reach a volume of 500 mL. HPLC analysis indicates that the solution contains 90.3 g (81.7%) of the epoxy alcohol product.

### Example 8

### Preparation of [3-(3-fluoro-phenyl)-oxiranyl]-methanol

A 1-L reactor equipped with a mechanical stirrer, thermocouple, and nitrogen inlet is charged with toluene (140 mL) and pre-activated molecular sieves powder (4A, 14 g). The resultant slurry was cooled to −35° C. To the reactor is then added a solution of D-(-)-diisopropyl tartrate (4.31 g, 18.4 mmol) in toluene (20 mL), followed by addition of titanium (IV) isopropoxide (3.74, 13.1 mmol). The temperature of the reaction mixture was maintained between −30° C. to −40° C. during the addition. To the reactor is then charged with a solution of 3-(3-fluoro-phenyl)-prop-2-en-1-ol (20 g, 131 mmol) in toluene (80 mL) while maintaining the temperature of the reaction mixture between −30° C. to −40° C. The reaction mixture is stirred at −35° C. for 30 minutes. To the reactor is then added a solution of cumene hydroperoxide (88% purity, 45.5 g, 263 mmol) while maintaining the temperature of the reaction mixture between −30° C. to −40° C. The reaction mixture is stirred at −35° C. for 16 hours. A solution of 30% sodium hydroxide saturated with sodium chloride (20 mL) is charged while maintaining temperature of the reaction mixture below −20° C. To the reaction mixture is then added a solution of sodium metabisulfite (13.7 g) in water (60 mL) while maintaining the reaction mixture temperature below 25° C. The biphasic mixture is stirred at room temperature for 1 hour. To the reaction mixture is added Celite (70 g) and the mixture is filtered. The filter cake is washed with toluene (2×50 mL). The filtrate is successively washed with 5% sodium bicarbonate (100 mL) and 10% brine (100 mL). The organic solution is then concentrated under vacuum to reach a volume of 100 mL.

### Example 9

### Preparation of [3-(3-fluoro-phenyl)-oxiranyl]-methanol

A 5-L jacketed reactor equipped with a mechanical stirrer, addition funnel, temperature probe, and nitrogen inlet. All equipment must be rigorously dry. The reactor was charged with D-(-)-DIPT (10.0 mL, 11.0 g, 46 mmol), 4-A, 5-um molecular sieves (49.3 g), dichloromethane (3 L). The flask was purged with nitrogen. The contents of the flask were cooled to 0° C. Titanium isopropoxide (9.34 g, 9.73 mL was added rapidly to the flask via an addition funnel. The reaction mixture was cooled to −20° C. A solution of allylic alcohol (100 g, 0.657 mol) in CH₂Cl₂ (300 mL) was added to the reaction mixture via an addition funnel while keeping the temperature below −20° C.

The reaction mixture was stirred at −20° C. for 10 minutes. A solution of TBHP in CH₂Cl₂ (188 mL, 5.7 M) was added to the reaction mixture via an addition funnel while maintaining the temperature between −20° C. to −25° C. The reaction mixture was stirred at −20° C. for 4 hours. Reaction progress was monitored by HPLC. A solution prepared from FeSO₄×6H₂O (217 g, 0.79 mol), citric acid monohydrate (72 g, 0.39 mol) and de-ionized water to the total volume of 660 mL, was chilled in an ice bath to 0° C.

The reaction mixture was quenched into the chilled solution of FeSO₄ and citric acid in water. The mixture was stirred for 30-60 minutes. The organic layer was checked for the presence of organic peroxides. The layers were separated. The aqueous phase was extracted with MTBE (2×200 mL). Combined organic solutions were cooled to 0° C. in an ice bath.

A 30% solution of NaOH (60 mL) in brine (prepared by dissolving 5 g of NaCl in a solution of NaOH (30.0 g) in 90 mL of water) was cooled in an ice bath to 0° C. and then added to the combined organic phases. The resulting mixture was stirred rapidly for 1-2 hours at 0° C. Water (300 mL) was added to the mixture. The two layers were separated. The aqueous layer was extracted with MTBE (2×250 mL). The combined organic layers were evaporated on rotary evaporator. HPLC analysis indicates that the solution contains 90.5 g (81.5%) of the epoxy alcohol product with chiral purity 95.6/4.4 and chemical purity 96.5 area %.

### Example 10

### Preparation of 7-fluoro-1-[(1S,2S)-1-(3-fluorophenyl)-2,3-dihydroxypropyl]-3,3-dimethyl-1,3-dihydro-2H-indol-2-one

To a suspension of 7-fluoro-3,3-dimethyl-oxindole (35 g, 0.195 mol) in N, N dimethylformamide (36 g, 0.49 mol) and toluene (200 mL) was added (1M/toluene) lithium bis(trimethylsilyl)amide (585 mL, 0.585 mol). To the resulting mixture was added a solution of (20%/toluene) [3-(3-fluoro-phenyl)-oxiranyl]-methanol (210 g, 0.253 mol) and titanium (IV) isopropoxide (72 g, 0.253 mol) in toluene (300 mL) at 5-10° C. The reaction mixture was stirred for 3-4 hours at 40-45° C. To the reaction mixture was added 37% HCl (460 g, 2.34 mol) and water (500 mL) at 20-25° C. to give a bi-phasic mixture. The organic layer was separated. The aqueous layer was extracted with toluene (1000 mL). The combined organic layers were washed with 1N NaOH (200 g), and then with 10% NaCl (200 g). The organic layer was concentrated via atmospheric distillation at 100-110° C. to a volume of (1800 mL). The concentrated solution was filtered through silica gel (150 g). The silica gel plug was rinsed with ethyl acetate (850 mL). The filtrate was concentrated via atmospheric distillation at 80-110° C. to reach a volume of (250 mL). The concentrate was cooled to 0-5° C. from 100-110° C. over 4 hours to crystallize. Solid was filtered, washed with heptane (150 mL), and oven-dried to give 50.6 g (74.7%) of a beige solid, 97.4% w/w purity by HPLC.

### Example 11

### Preparation of 7-fluoro-1-[(1 S,2R)-1-(3-fluorophenyl)-2-hydroxy-3-(methyl amino)propyl]-3,3-dimethyl-1,3-dihydro-2H-indol-2-one

To the solution of the diol (52 g, 0.144 mol) in MeCN (500 mL) was added Bu₂SnO (0.39 g, 1.44 mmol) and TsCl (28.8 g, 0.151 mol). To the resulting solution was added Et₃N (29 g, 0.288 mol) dropwise at 0-5° C. The reaction was stirred for 1 hour at 0-5° C. until the tosylation was complete by HPLC. To the reaction containing the mono-tosylate was added a solution of NaOH (58 g, 0.72 mol) in water (400 mL) at 0° C. At the end of the epoxide formation, toluene (800 mL) and NaCl (25 g) in water (150 mL) were added to form a bi-phasic reaction mixture. The two layers were separated. The organic layer was washed with 37% w/w HCl (56 g) in water (256 mL) followed by NaCl (50 g) in water (300 mL). The organic layer was diluted with toluene (700 mL) and concentrated to a volume of about 900 mL. The resulting concentrated solution was filtered through a silica gel (200 g) plug. The silica gel plug was eluted with toluene (1.5 L). The combined filtrate was concentrated under vacuum to about 300 mL. Methylamine in EtOH (33 weight %, 245 mL, 2.0 mol) and Ca(OTf)₂ (15 g, 43 mmol) were added to the toluene solution. The reaction mixture was stirred at 20-25° C. for 12 hours then concentrated via vacuum distillation to about 200 mL. MTBE (500 mL) and water (500 mL) were added. The two layers were separated. 37% w/w HCl (160 g,) in water (340 g) was added to the organic layer. Stirred and the two layers were separated. The aqueous organic layer was washed with MTBE (500 mL). To the acidic aqueous layer was charged MTBE (500 mL) then the mixture was cooled to 0-5° C. and basified with NaOH (50% w/w, 150 g, 100 mL). Reaction mixture was stirred for 20 minutes then the two layers were separated. The organic layer was washed with 15% NaCl (170 mL) then concentrated to about 250 mL via atmospheric distillation. To the MTBE concentrate was added EtOH (2B) (150 mL) followed by HCl (5.7 N in EtOH, 45 mL, 0.26 mol). The mixture was stirred at 20 to 25° C. for a minimum of 2 hours and then cooled to 0 to 5° C. over 1 hour. The suspension was filtered and washed with MTBE (50 mL) to give 26 g (45%) of an off-white solid.

### Example 12

### Preparation of (2E)-3-(3,5-difluorophenyl)prop-2-en-1-ol

A 5-L reactor equipped with a mechanical stirrer, thermocouple, and nitrogen inlet was charged with MeOH (1.40 L), 3,5-difluorocinnamic acid (0.20 kg, 1.09 mol) and p-TSA (0.0207 kg, 0.109 mol) at 20° C. to 25° C. The suspension was refluxed at 65° C. to 68° C. for 4-6 hours. The mixture was concentrated via atmospheric distillation to reach a volume of about 700 mL. Methanol was then chased off by adding toluene (1.8 L) and was further concentrated to a solution (about 1.5 L). The reaction was cooled to 50° C. to 55° C. then washed successively with 5% aqueous NaHCO₃ (1.5 L) and water (1.5 L). The organic mixture was concentrated via atmospheric distillation to a minimum volume of about 1.5 L. KF 0.17%.

A 3-L reactor equipped with a mechanical stirrer, thermocouple, and nitrogen inlet was charged with diisobutylaluminum hydride 25% w/w in toluene (1.42 kg, 1.68 L, 2.31 mol). The solution was cooled to −25° C. To the reactor was then added using FMI pump a solution of 3-(3,5-difluoro-phenyl)-acrylic acid methyl ester (1.4 L, 1.09 mol) in toluene while maintaining the internal temperature between −15° C. to −8° C. The reaction mixture was stirred at that temperature for 60 minutes then quenched into a 5-L reactor with a solution of concentrated HCl (0.40 L, 0.48 kg; 4.87 mol) in water (0.70 kg) while maintaining the internal temperature at 40° C. to 45° C. The biphasic mixture was separated. The lower aqueous layer was washed with toluene (0.34 kg, 0.40 L). The combined organic phase was successively washed with a 5% aqueous solution of sodium bicarbonate (0.70 L) and 10% brine (0.70 L). The organic solution was concentrated via atmospheric distillation to reach a volume of 0.386 Kg, about 500 mL. HPLC analysis indicates that the solution contains 170 g, 91% yield of (2E)-3-(3,5-difluorophenyl)prop-2-en-1-ol. Al: 1 ppm, KF: 0.12%, 99.8% area HPLC purity.

### Example 13

### Preparation of [(2R,3R)-3-(3,5-difluorophenyl)oxiran-2-yl]-methanol

A 3-L reactor equipped with a mechanical stirrer, thermocouple, and nitrogen inlet was charged with toluene (100 mL) and pre-activated molecular sieves powder (4A, 70 g). The resultant slurry was cooled to −35° C. To the reactor was then added a solution of D-(-)-diisopropyl tartrate (19.3 g, 0.082 mol) in toluene (25 mL), followed by addition of titanium (IV) isopropoxide (16.7 g, 0.059 mol). The temperature of the reaction mixture was maintained between −30° C. to −40° C. during the addition. To the reactor was then added a solution of 3-(3,5-difluoro-phenyl)-prop-2-en-1-ol (100 g, 0.588 mol) in toluene (250 mL) while maintaining the temperature of the reaction mixture between −30° C. to −40° C. The reaction mixture was stirred at −35° C. for 30 min. To the reactor was then added a solution of 5.5 M tert-butyl hydroperoxide in decane (173 g, 1.18 mol) while maintaining the temperature of the reaction mixture between −30° C. to −40° C. The reaction mixture was stirred at −35° C. for 6 hours, followed by 8 hours at −25° C. The reaction mixture was warmed to room temperature and filtered through a thin bed of celite (25 g). The filter cake was washed with toluene (2×200 mL). The combined filtrate and washes were cooled to 0° C. and a solution of 30% sodium hydroxide saturated with sodium chloride (100 mL) was then added. The reaction mixture is stirred at 0° C. for 3 h. To the reaction mixture was then added a solution of sodium metabisulfite (61.5 g) and citric acid (44.5 g) in water (600 mL). The biphasic mixture was stirred at room temperature for 1 hour and the phases were separated. The organic phase was successively washed with a 5% sodium bicarbonate (500 mL) and 10% brine (500 mL). The organic solution was then concentrated under vacuum to reach a volume of about 400 mL. A small portion of the concentrate was taken out for seed generation at 25-30° C. To the suspension was then charged 3 volume parts of heptane (300-400 mL). The mixture was cooled to 5-10° C. then filtered to give 71.3 g, 65% yield of [(2R,3R)-3-(3,5-difluorophenyl)oxiran-2-yl]-methanol as an off-white solid with chiral purity 94% ee, mp: 48-50° C.

### Example 14

### Preparation of 1-[(1S,2S)-1-(3,5-difluorophenyl)-2,3-dihydroxypropyl]-7-fluoro-3,3-dimethyl-1,3-dihydro-2H-indol-2-one

To a suspension of dimethyl oxidole (68 g of 74% strength crude, 280 mmol) in DMF (51 g, 700 mmol.) and toluene (200 mL), a toluene solution of (Me₃Si)₂NLi (840 mL, 1 M, 840 mmol) was added dropwise while keeping the mixture below 10° C. to give a dark solution. A solution of epoxy alcohol (76 g of 85% strength, 350 mmol) and Ti(OiPr)₄ (103 g, 360 mmol) in toluene (400 mL) was added to the above dark solution at below 10° C. The reaction mixture was stirred for 20 hours at 20° C. before cooling to 0° C. A solution of HCl (660 g, 37% in water) in water (750 g) was added at below 20° C. to give a bi-phasic mixture. The two layers were separated. The organic layer was washed with NaOH (400 mL, 0.7 N in water, 280 mmol), and brine (230 g). The organic layer was filtered through a silica gel (150 g) plug. The silica gel plug was rinsed with EtOAc (1100 mL). The filtrate was concentrated in vacuo at 50° C. to a volume of 240 mL. This concentrate was diluted with CH₃CN (300 mL) to give 1-[(1S,2S)-1-(3,5-difluorophenyl)-2,3-dihydroxypropyl]-7-fluoro-3,3-dimethyl-1,3-dihydro-2H-indol-2-one as a CH₃CN solution, 431 g of a 20.8% strength solution, yield: 88%.

### Example 15

### Preparation of 1-[(1S,2R)-1-(3,5-difluorophenyl)-2-hydroxy-3-(methylamino)propyl]-7-fluoro-3,3-dimethyl-1,3-dihydro-2H-indol-2-one

To the solution of 1-[(1S,2S)-1-(3,5-difluorophenyl)-2,3-dihydroxypropyl]-7-fluoro-3,3-dimethyl-1,3-dihydro-2H-indol-2-one in acetonitrile (394 g of 20.8% strength solution, 224 mmol) at 20° C., tosyl chloride (56 g, 269 mmol) and Bu₂SnO (1.4 g 5.6 mmol) were added. The reaction mixture was cooled to 5° C., and then Et₃N (45 g, 448 mmol) was added dropwise. The reaction mixture was stirred for about 1 hour at 20° C. until tosylation is complete.

A solution of NaOH (90 g of 50% w/w solution in water, 1120 mmol) in water (492 g) was added at 5° C. The reaction mixture was stirred for 1 hour. Toluene (1312 mL) was added to the reaction mixture to give a bi-phasic mixture. The organic layer was separated and washed with HCl (44 g of 37% solution in water, 448 mmol) in water (320 mL) then with brine (400 mL). The organic layer was then concentrated to a volume of (400 mL) under vacuum keeping the temperature below 50° C. The concentrate was diluted with toluene (1120 mL). The resulting solution was filtered through a silica gel (320 g) plug. The silica gel plug was eluted with toluene (2400 mL). The filtrate was concentrated to a volume of 400 mL in vacuo keeping the temperature below 50° C.

Methanol (1200 mL) was charged to the mixture then concentrated down to about 400 mL in vacuo while keeping temperature below 50° C. To the concentrate was added methanol (1600 mL) and methylamine (252 g of 33 wt % solution in ethanol, 2688 mmol.). The reaction mixture was stirred for 20 hours at 40° C. until the aminolysis is complete. The mixture was concentrated down to about 400 mL in vacuo. Toluene (960 mL) was added to the concentrate. The mixture was concentrated in vacuo down to about 400 mL.

HCl (40 g of 5N solution in isopropanol, 224 mmol.) in IPA was added to the mixture. Stirred at 20° C. for 2 hours. The resulting slurry was filtered then dissolved in acetone (1230 mL) at 40° C. Heptane (1640 mL) was added. The resulting solution was concentrated at 70° C. to a volume of (1230 mL). The resulting slurry was filtered and dried for 18 hours at 55° C. to give 46.5 g, 50% overall yield of 1-[(1S,2R)-1-(3,5-difluorophenyl)-2-hydroxy-3-(methylamino)propyl]-7-fluoro-3,3-dimethyl-1,3-dihydro-2H-indol-2-one as a white solid.

### Example 16

### Preparation of 7-fluoro-1-{(1S)-(3-fluorophenyl)[(2S)-oxiran-2-yl]methyl}-3,3-dimethyl-1,3-dihydro-2H-indol-2-one

Diethyl-azodicarboxylate (100 g, 572 mmol) was added dropwise to a solution of 1-[(1S,2S)-1-(3,5-difluorophenyl)-2,3-dihydroxypropyl]-7-fluoro-3,3-dimethyl-1,3-dihydro-2H-indol-2-one (90 g, 260 mmol) and Ph₃P (129 g, 520 mmol) in toluene (1042 mL) at 25° C. The mixture was stirred for 1 hour at 80° C. Ph₃P (7 g, 26 mmol) was added to the mixture at 80° C. The mixture was stirred for 8 hours at 80° C. Diethyl-azodicarboxylate (9 g, 52 mmol) was added to the mixture at 80° C. The mixture was stirred for about 2 hours at 80° C. until the reaction is complete. Heptane (3120 mL) was added to the reaction mixture at 80° C. The mixture was cooled to 10° C. and then filtered through a silica gel (720 g) plug. The filtrate was discarded. The silica gel plug was rinsed with a solution of ethyl acetate (1100 mL) in heptane (3300 mL). The filtrate was concentrated to dryness at 50° C. to give 56 g, 80% purity, 52% yield of 7-fluoro-1-{(1S)-(3-fluorophenyl)[(2S)-oxiran-2-yl]methyl}-3,3-dimethyl-1,3-dihydro-2H-indol-2-one.

### Example 17

### Preparation of 7-fluoro-1-[(1S,2R)-1-(3-fluorophenyl)-2-hydroxy-3-(methyl amino)propyl]-3,3-dimethyl-1,3-dihydro-2H-indol-2-one

In a flask with 7-fluoro-1-[(1S,2S)-1-(3-fluorophenyl)-2,3-dihydroxypropyl]-3,3-dimethylindolin-2-one (10 g, 0.0288 mol) and para-toluenesulfonic acid (pTSA) (0.0438 g, 0.023 mol) in THF (50 mL), trimethyl orthoacetate (4.15 g, 4.3 mL, 0.0346 moles) was added dropwise. The amber color solution was stirred at room temperature for 1 hour. The reaction mixture was concentrated to oil then THF (50 mL) was added. Cooled to 0° C. to 5° C. then acetyl bromide (8.50 g, 0.0692 mol) was added. The resulting mixture was stirred at room temperature for 3 to 4 hours then concentrated to oil and charged with THF (25 mL) and EtOH 2B (25 mL) followed by K₂CO₃—325 (39.8 g, 0.288 mol). The mixture was stirred at room temperature then the mixture was concentrated in vacuo to oil. MTBE (100 mL) and H₂O (170 mL) were added to dissolve the oil. The two layers were separated. The aqueous layer was extracted with MTBE (2×100 mL). The combined organic layer was concentrated to oil then 33% solution of methylamine in ethanol (15 eq.) was added and stirred at room temperature. At the completion of the reaction, the mixture was concentrated to oil. MTBE 100 mL) and H₂O (100 mL) were added. The two layers were separated. The organic layer was extracted with 37% concentrated HCl (30.7 g) in H₂O (65 g). The lower aqueous layer was extracted with MTBE (100 mL) then cooled to 0-5° C. MTBE (100 mL) and a solution of 50% NaOH (30 g) in H₂O (30 g) were added to the aqueous layer. The mixture was stirred for 20 minutes at room temperature and the layers were separated. The aqueous layer was back extracted with MTBE (50 mL). The combined organic layer was washed with a 15% NaCl (23 mL) solution. The organic layer was concentrated to give as oil (8.4 g, about 90% by LC/MS, 60% yield).

When ranges are used herein for physical properties, such as molecular weight, or chemical properties, such as chemical formulae, all combinations and subcombinations of ranges specific embodiments therein are intended to be included.

The disclosures of each patent, patent application and publication cited or described in this document are hereby incorporated herein by reference, in their entireties.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

What is claimed:

1. A process, comprising the step of:
a. coupling a compound of formula IV: 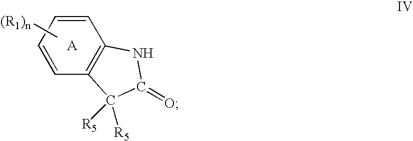
or a metal salt thereof;
with a compound of formula II: 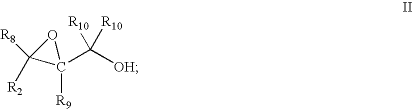
to form a diol compound of formula V: 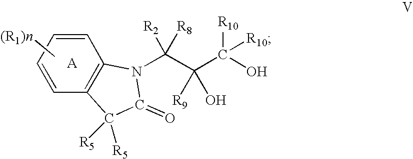
wherein said coupling is carried out in the presence of:
an optional Lewis acid catalyst;
a solvent composition comprising at least one polar, aprotic solvent; and
an excess of a strong non-nucleophilic base selected from the group consisting of RₓRₓ—N-M, R_{y}—O-M, and R_{y}—Mg—X;
where:
each Rₓ is independently alkyl substituted with 0-3 R₁, aryl substituted with 0-3 R₁, or (R_{z})₃Si;
or said Rₓ groups, together with the N atom to which they are attached, form a cyclic amine;
R_{y} is alkyl substituted with 0-3 R₁;
R_{z} is R₁;
M is Na, Li, or K;
X is Cl, Br, or I;
provided that said strong non-nucleophilic base is other than sodium t-butoxide;
wherein:
R₁ is, independently at each occurrence, alkyl, alkoxy, halo, CF₃, OCF₃, arylalkyloxy substituted with 0-3 R₁₁, aryloxy substituted with 0-3 R₁₁, aryl substituted with 0-3 R₁₁, heteroaryl substituted with 0-3 R₁₁, hydroxy, alkanoyloxy, nitro, nitrile, alkenyl, alkynyl, alkylsulfoxide, phenylsulfoxide substituted with 0-3 R₁₁, alkylsulfone, phenylsulfone substituted with 0-3 R₁₁, alkylsulfonamide, phenylsulfonamide substituted with 0-3 R₁₁, heteroaryloxy substituted with 0-3 R₁₁, heteroarylmethyloxy substituted with 0-3 R₁₁, alkylamido, or arylamido substituted with 0-3 R₁₁; or two adjacent R₁ also represent methylenedioxy;
R₂ is aryl substituted with 0-3 R₁ or heteroaryl substituted with 0-3 R₁;
R₅ is, independently at each occurrence, H, C₁-C₄ alkyl, aryl substituted with 0-3 R₁, or cyano; or the two R₅ form a carbocyclic ring of 3-7 carbons;
R₈ is H, or C₁-C₄ alkyl;
R₉ is H, or C₁-C₄ alkyl;
R₁₀ is, independently at each occurrence, H, or C₁-C₄ alkyl;
R₁₁ is alkyl, alkoxy, halo, CF₃, OCF₃, hydroxy, alkanoyloxy, nitro, nitrile, alkenyl, alkynyl, alkylsulfoxide, alkylsulfone, alkylsulfonamide, or alkylamido; or two adjacent R₁₁ also represent methylenedioxy;
n is an integer from 0 to 4; and
wherein 1-3 carbon atoms in ring A may optionally be replaced with N.

2. A process according to claim 1, wherein:
said strong non-nucleophilic base is a base selected from the group consisting of lithium hexamethyldisilazide (LHMDS), potassium hexamethyldisilazide (KHMDS), lithium diisopropylamide (LDA), potassium butoxide (KOtBu), and combinations thereof.

3. A process according to claim 1, wherein:
said strong non-nucleophilic base is lithium hexamethyldisilazide (LHMDS).

4. A process according to claim 1, wherein:
said Lewis acid is titanium (IV) isopropoxide.

5. A process according to claim 1, wherein:
said solvent composition comprises dimethylformamide (DMF).

6. A process according to claim 5, wherein:
said solvent composition further comprises tetrahydrofuran (THF) or toluene.

7. A process according to claim 1, further comprising the step of:
b. selectively activating the terminal hydroxy group of said diol compound of formula V with a compound of the formula (R₁₂SO₂)₂O or R₁₂SO₂Z with or without the use of catalyst in the presence of an optional base in an inert solvent to form a compound of formula Va: 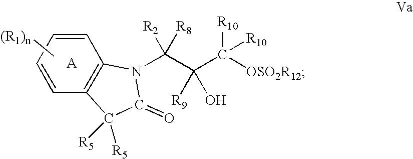
wherein:
Z is Cl or Br; and
R₁₂ is alkyl substituted with 0-3 R₁ or aryl substituted with 0-3 R₁.

8. A process according to claim 7, wherein:
R₁₂ is methyl, ethyl, propyl, butyl, trifluoromethyl (triflate), phenyl, or benzyl, any of which may be optionally substituted with one or more substituents selected from the group consisting of C₁-C₄ alkyl, C₁-C₄ alkoxy, halo, and nitro.

9. A process according to claim 8, wherein:
R₁₂ is p-tolyl, methyl, brosyl, p-methoxyphenyl, p-ethoxyphenyl, pentafluorophenyl, or 2,4,6-triisopropyl.

10. A process according to claim 7, further comprising the step of:
c. converting said compound of formula Va in the presence of a base and an optional phase transfer catalyst to a compound of formula VI:

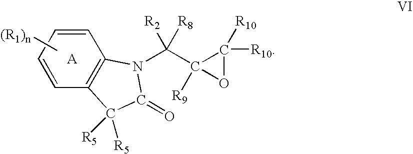

11. A process according to claim 10, wherein:
said base is aqueous sodium hydroxide (NaOH), aqueous potassium hydroxide (KOH), aqueous potassium carbonate (K₂CO₃), or mixtures thereof.

12. A process according to claim 10, wherein:
said optional phase transfer catalyst is (R₁₃)₄NX′, where:
R₁₃ is alkyl substituted with 0-3 R₁ or aryl substituted with 0-3 R₁; and
X′ is a counterion.

13. A process according to claim 12, wherein:
said optional phase transfer catalyst is BU₄NCl.

14. A process according to claim 7, further comprising the step of:
c. treating said compound of formula V with phosphine and dialkyl azodicarboxylate in inert solvent to form a compound of formula VI:

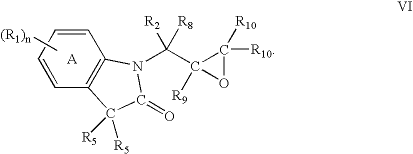

15. A process according to claim 10 or 14, further comprising the step of:
d. reacting said compound of formula VI with NHR₄R₄ with optional Lewis acid catalyst in an optional polar solvent to form a compound of formula I: 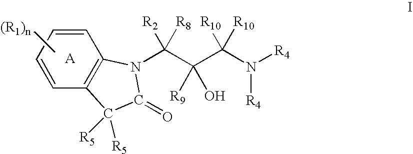
wherein:
R₄ is, independently at each occurrence, H, C₁-C₄ alkyl, arylalkyl, heteroarylmethyl, cycloheptylmethyl, cyclohexylmethyl, cyclopentylmethyl, or cyclobutylmethyl; and
with respect to the compound of formula I, R₁₀ and R₄, together with the nitrogen to which R₄ is attached, form a nitrogen-containing ring containing 3 to 6 carbons.

16. A process according to claim 15, wherein:
NHR₄R₄ is NH₂CH₃.

17. A process according to claim 15, further comprising the step of:
e. forming a pharmaceutically acceptable salt of said compound of formula I.

18. A process according to claim 17, wherein:
said pharmaceutically acceptable salt is a hydrochloride salt.

19. A process according to claim 15, wherein:
said steps b, c, and d are telescoped.

20. A process according to claim 1, wherein:
said compound of formula II is formed from an allylic alcohol of formula III: 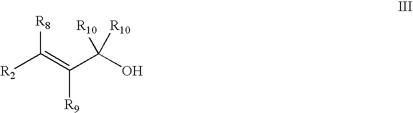
by reacting said compound of formula III with a homochiral diester of a tartaric acid and a hydroperoxide, in the presence of a metal catalyst in optional inert solvent.

21. A process according to claim 20, wherein:
said reaction of said compound of formula III is quenched with a reducing agent and optional citric acid.

22. A process according to claim 20, wherein:
said allylic alcohol of formula III is formed by reducing a compound of formula VIII: 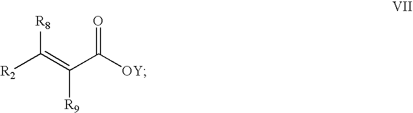
wherein:
Y is alkyl substituted with 0-3 R₁, aryl substituted with 0-3 R₁, or heteroaryl substituted with 0-3 R₁;

23. A process according to claim 22, wherein:
Y is C₁-C₄ alkyl.

24. A process according to claim 22, wherein:
said compound of formula VII is formed by esterifying a compound of formula VIII: 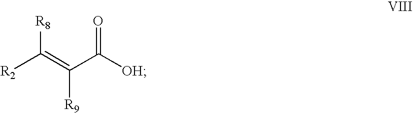
or a salt thereof.

25. A process according to claim 1, wherein:
said compound of formula IV is formed from a compound of formula IX: 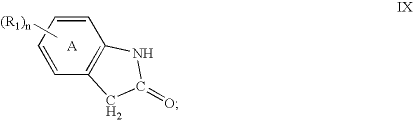
or a salt thereof.

26. A process according to claim 25, wherein:
said compound of formula IX is formed by reducing a compound of formula X:

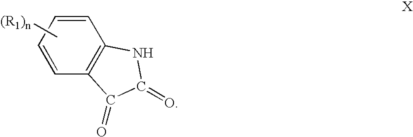

27. A process according to claim 15, wherein:
said compound of formula I is a compound of formula I*:

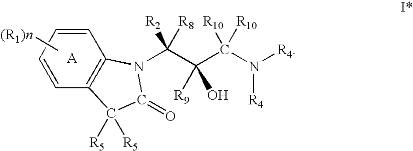

28. A process according to claim 27, wherein:
said compound of formula I is

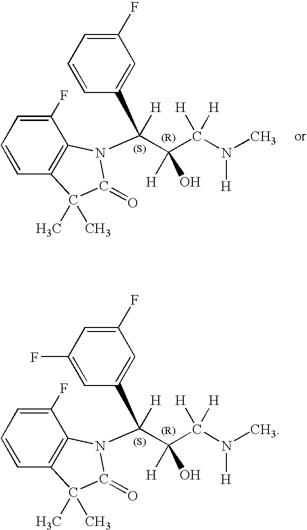

29. A process according to claim 17 or 18, wherein:
said compound of formula I is

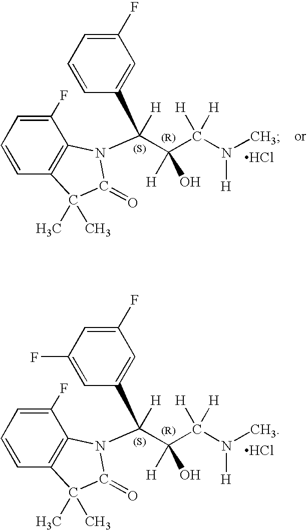

30. A process according to claim 1, wherein:
said compound of formula II is a compound of formula II*:

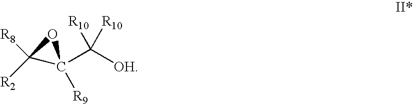

31. A process according to claim 1, wherein:
said compound of formula V is a compound of formula V*:

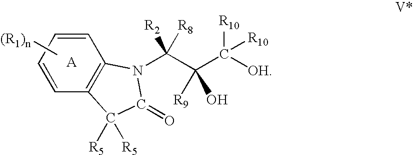

32. A process according to claim 10, wherein:
said compound of formula VI is a compound of formula VI*:

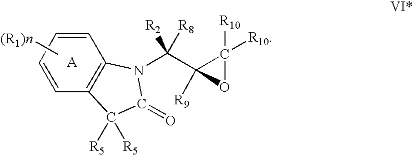

33. A process according to claim 1, wherein:
R₁ is, independently at each occurrence, halo.

34. A process according to claim 33, wherein:
R₁ is F.

35. A process according to claim 1, wherein:
R₂ is aryl substituted with R₁.

36. A process according to claim 35, wherein:
R₂ is phenyl substituted with F.

37. A process according to claim 36, wherein:
R₂ is m-fluorophenyl.

38. A process according to claim 15, wherein:
R₄ is, independently at each occurrence, H or C₁ alkyl.

39. A process according to claim 1, wherein:
R₅ is C₁ alkyl.

40. A process according to claim 1, wherein:
R₈ is H.

41. A process according to claim 1, wherein:
R₉ is H.

42. A process according to claim 1, wherein:
R₁₀ is H.

43. A process according to claim 1, wherein:
n is 1.

44. A process, comprising the step of:
aa. transesterifying a diol compound of formula V: 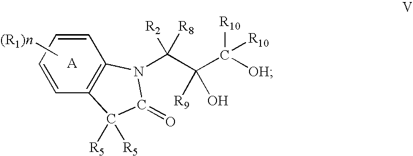
with a trialkyl orthoacetate in the presence of a catalytic amount of an acid or an acid catalyst to form a cyclic orthoester compound of formula XI: 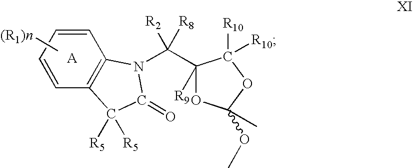
wherein:
R₁ is, independently at each occurrence, alkyl, alkoxy, halo, CF₃, OCF₃, arylalkyloxy substituted with 0-3 R₁₁, aryloxy substituted with 0-3 R₁₁, aryl substituted with 0-3 R₁₁, heteroaryl substituted with 0-3 R₁₁, hydroxy, alkanoyloxy, nitro, nitrile, alkenyl, alkynyl, alkylsulfoxide, phenylsulfoxide substituted with 0-3 R₁₁, alkylsulfone, phenylsulfone substituted with 0-3 R₁₁, alkylsulfonamide, phenylsulfonamide substituted with 0-3 R₁₁, heteroaryloxy substituted with 0-3 R₁₁, heteroarylmethyloxy substituted with 0-3 R₁₁, alkylamido, or arylamido substituted with 0-3 R₁₁; or two adjacent R₁ also represent methylenedioxy;
R₂ is aryl substituted with 0-3 R₁ or heteroaryl substituted with 0-3 R₁;
R₅ is, independently at each occurrence, H, C₁-C₄ alkyl, aryl substituted with 0-3 R₁, or cyano; or the two R₅ form a carbocyclic ring of 3-7 carbons;
R₈ is H, or C₁-C₄ alkyl;
R₉ is H, or C₁-C₄ alkyl;
R₁₀ is, independently at each occurrence, H, or C₁-C₄ alkyl;
R₁₁ is alkyl, alkoxy, halo, CF₃, OCF₃, hydroxy, alkanoyloxy, nitro, nitrile, alkenyl, alkynyl, alkylsulfoxide, alkylsulfone, alkylsulfonamide, or alkylamido; or two adjacent R₁₁ also represent methylenedioxy;
n is an integer from 0 to 4; and
wherein 1-3 carbon atoms in ring A may optionally be replaced with N.

45. A process according to claim 44, further comprising the step of:
bb. reacting said cyclic orthoester compound of formula XI with a trimethylsilyl-X″ or acetyl-X″ to form a halohydrin ester of formula XII: 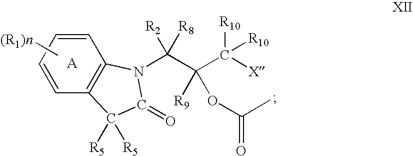
wherein:
X″ is Cl, Br, or I.

46. A process according to claim 44, further comprising the step of:
bb. converting said cyclic orthoester compound of formula XI to a halohydrin ester of formula XII: 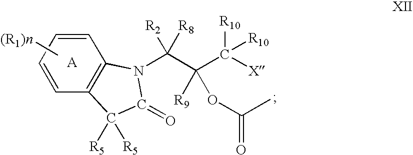
wherein:
X″ is Cl, Br, or I.
and then converting said halohydrin ester compound of formula XII to a compound of formula VI:

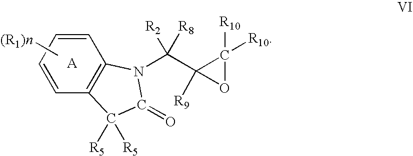

47. A process according to claim 45 or 46, further comprising the step of:
cc. treating said halohydrin ester of formula XII with base in a polar solvent to form a compound of formula VI:

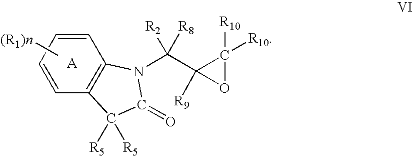

48. A process according to claim 47, wherein:
said steps aa, bb, and cc are telescoped.

49. A process according to claim 46, further comprising the step of:
dd. reacting said compound of formula VI with NHR₄R₄ and optional Lewis acid catalyst in an optional polar solvent to form a compound of formula I: 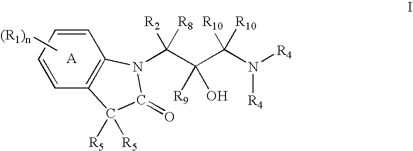
wherein:
R₄ is, independently at each occurrence, H, C₁-C₄ alkyl, arylalkyl, heteroarylmethyl, cycloheptylmethyl, cyclohexylmethyl, cyclopentylmethyl, or cyclobutylmethyl; and
with respect to the compound of formula I, R₁₀ and R₄, together with the nitrogen to which R₄ is attached, form a nitrogen-containing ring containing 3 to 6 carbons.

50. A process according to claim 45, further comprising the step of:
ee. reacting said halohydrin ester of formula XII with NHR₄R₄ in an optional polar solvent to form a compound of formula I: 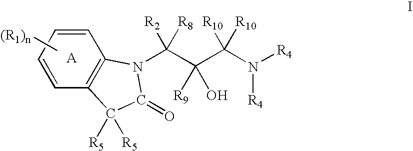
wherein:
R₄ is, independently at each occurrence, H, C₁-C₄ alkyl, arylalkyl, heteroarylmethyl, cycloheptylmethyl, cyclohexylmethyl, cyclopentylmethyl, or cyclobutylmethyl; and
with respect to the compound of formula I, R₁₀ and R₄, together with the nitrogen to which R₄ is attached, form a nitrogen-containing ring containing 3 to 6 carbons.

51. An isolated, solid form of a compound of formula V: 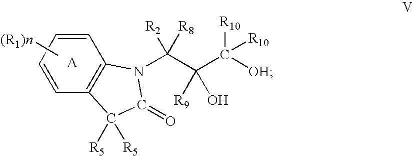
wherein:
R₁ is, independently at each occurrence, alkyl, alkoxy, halo, CF₃, OCF₃, arylalkyloxy substituted with 0-3 R₁₁, aryloxy substituted with 0-3 R₁₁, aryl substituted with 0-3 R₁₁, heteroaryl substituted with 0-3 R₁₁, hydroxy, alkanoyloxy, nitro, nitrile, alkenyl, alkynyl, alkylsulfoxide, phenylsulfoxide substituted with 0-3 R₁₁, alkylsulfone, phenylsulfone substituted with 0-3 R₁₁, alkylsulfonamide, phenylsulfonamide substituted with 0-3 R₁₁, heteroaryloxy substituted with 0-3 R₁₁, heteroarylmethyloxy substituted with 0-3 R₁₁, alkylamido, or arylamido substituted with 0-3 R₁₁; or two adjacent R₁ also represent methylenedioxy;
R₂ is aryl substituted with 0-3 R₁ or heteroaryl substituted with 0-3 R₁;
R₅ is, independently at each occurrence, H, C₁-C₄ alkyl, aryl substituted with 0-3 R₁, or cyano; or the two R₅ form a carbocyclic ring of 3-7 carbons;
R₈ is H, or C₁-C₄ alkyl;
R₉ is H, or C₁-C₄ alkyl;
R₁₀ is, independently at each occurrence, H, or C₁-C₄ alkyl;
R₁₁ is alkyl, alkoxy, halo, CF₃, OCF₃, hydroxy, alkanoyloxy, nitro, nitrile, alkenyl, alkynyl, alkylsulfoxide, alkylsulfone, alkylsulfonamide, or alkylamido; or two adjacent R₁₁ also represent methylenedioxy;
n is an integer from 0 to 4; and
wherein 1-3 carbon atoms in ring A may optionally be replaced with N.

52. A compound according to claim 51, wherein:
R₁ is, independently at each occurrence, halo.

53. A compound according to claim 52, wherein:
R₁ is F.

54. A compound according to claim 51, wherein:
R₂ is aryl substituted with at least one R₁.

55. A compound according to claim 54, wherein:
R₂ is phenyl substituted with at least one F.

56. A compound according to claim 55, wherein:
R₂ is m-fluorophenyl or 3,5-difluorophenyl.

57. A compound according to claim 51, wherein:
R₅ is C₁ alkyl.

58. A compound according to claim 51, wherein:
R₈ is H.

59. A compound according to claim 51, wherein:
R₉ is H.

60. A compound according to claim 51, wherein:
R₁₀ is H.

61. A compound according to claim 51, wherein:
n is 1.

62. A compound of formula VI: 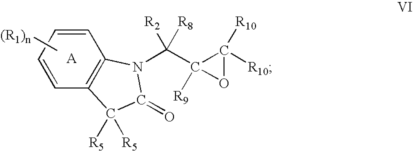
wherein:
R₁ is, independently at each occurrence, alkyl, alkoxy, halo, CF₃, OCF₃, arylalkyloxy substituted with 0-3 R₁₁, aryloxy substituted with 0-3 R₁₁, aryl substituted with 0-3 R₁₁, heteroaryl substituted with 0-3 R₁₁, hydroxy, alkanoyloxy, nitro, nitrile, alkenyl, alkynyl, alkylsulfoxide, phenylsulfoxide substituted with 0-3 R₁₁, alkylsulfone, phenylsulfone substituted with 0-3 R₁₁, alkylsulfonamide, phenylsulfonamide substituted with 0-3 R₁₁, heteroaryloxy substituted with 0-3 R₁₁, heteroarylmethyloxy substituted with 0-3 R₁₁, alkylamido, or arylamido substituted with 0-3 R₁₁; or two adjacent R₁ also represent methylenedioxy;
R₂ is aryl substituted with 0-3 R₁ or heteroaryl substituted with 0-3 R₁;
R₅ is, independently at each occurrence, H, C₁-C₄ alkyl, aryl substituted with 0-3 R₁, or cyano; or the two R₅ form a carbocyclic ring of 3-7 carbons;
R₈ is H, or C₁-C₄ alkyl;
R₉ is H, or C₁-C₄ alkyl;
R₁₀ is, independently at each occurrence, H, or C₁-C₄ alkyl;
R₁₁ is alkyl, alkoxy, halo, CF₃, OCF₃, hydroxy, alkanoyloxy, nitro, nitrile, alkenyl, alkynyl, alkylsulfoxide, alkylsulfone, alkylsulfonamide, or alkylamido; or two adjacent R₁₁ also represent methylenedioxy;
n is an integer from 0 to 4; and
wherein 1-3 carbon atoms in ring A may optionally be replaced with N.

63. A compound according to claim 62, wherein:
R₁ is, independently at each occurrence, halo.

64. A compound according to claim 63, wherein:
R₁ is F.

65. A compound according to claim 62, wherein:
R₂ is aryl substituted with at least one R₁.

66. A compound according to claim 65, wherein:
R₂ is phenyl substituted with at least one F.

67. A compound according to claim 66, wherein:
R₂ is m-fluorophenyl or 3,5-difluorophenyl.

68. A compound according to claim 62, wherein:
R₅ is C₁ alkyl.

69. A compound according to claim 62, wherein:
R₈ is H.

70. A compound according to claim 62, wherein:
R₉ is H.

71. A compound according to any one of claims 62 to 70, wherein:
R₁₀ is H.

72. A compound according to claim 62, wherein:
n is 1.

73. A compound according to claim 62 selected from the group consisting of:

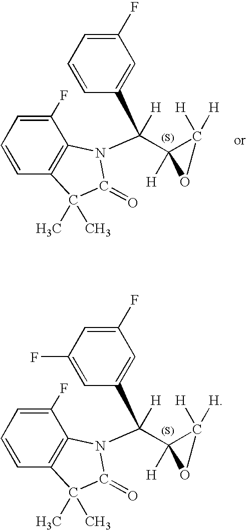

74. A product produced by the process of claim 15.

75. A product produced by the process of claim 17.

76. A composition, comprising:
a compound of formula I; and
less than about 35% by weight, based on the total weight of the composition, of a compound of formula I′: 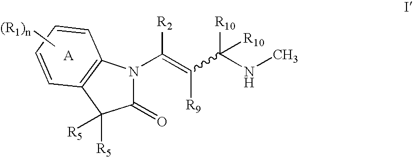
R₁ is, independently at each occurrence, alkyl, alkoxy, halo, CF₃, OCF₃, arylalkyloxy substituted with 0-3 R₁₁, aryloxy substituted with 0-3 R₁₁, aryl substituted with 0-3 R₁₁, heteroaryl substituted with 0-3 R₁₁, hydroxy, alkanoyloxy, nitro, nitrile, alkenyl, alkynyl, alkylsulfoxide, phenylsulfoxide substituted with 0-3 R₁₁, alkylsulfone, phenylsulfone substituted with 0-3 R₁₁, alkylsulfonamide, phenylsulfonamide substituted with 0-3 R₁₁, heteroaryloxy substituted with 0-3 R₁₁, heteroarylmethyloxy substituted with 0-3 R₁₁, alkylamido, or arylamido substituted with 0-3 R₁₁; or two adjacent R₁ also represent methylenedioxy;
R₂ is aryl substituted with 0-3 R₁ or heteroaryl substituted with 0-3 R₁;
R₅ is, independently at each occurrence, H, C₁-C₄ alkyl, aryl substituted with 0-3 R₁, or cyano; or the two R₅ form a carbocyclic ring of 3-7 carbons;
R₉ is H, or C₁-C₄ alkyl;
R₁₀ is, independently at each occurrence, H, or C₁-C₄ alkyl;
R₁₁ is alkyl, alkoxy, halo, CF₃, OCF₃, hydroxy, alkanoyloxy, nitro, nitrile, alkenyl, alkynyl, alkylsulfoxide, alkylsulfone, alkylsulfonamide, or alkylamido; or two adjacent R₁₁ also represent methylenedioxy;
n is an integer from 0 to 4;
wavy line represents both stereochemical configurations between the carbons to which R₉ and R₁₀ are attached; and
wherein 1-3 carbon atoms in ring A may optionally be replaced with N.
